(19) United States
(12) Patent Application Publication
(10) Pub. No.: US 2004/0102633 A1
(43) Pub. Date: May 27, 2004

(54) Novel compounds

(76) Inventors: Carlo Farina, Baranzate di Bollate (IT); Giuseppe Giardina, Baranzate di Bollate (IT); Mario Grugni, Baranzate di Bollate (IT); Guy Marguerite Marie Gerard Nadler, Rennes (FR)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/433,595

(22) PCT Filed: Nov. 26, 2001
(86) PCT No.: PCT/EP01/13832

(30) Foreign Application Priority Data
Nov. 28, 2000 (GB) 0028964.5

(51) Int. Cl. C 07D 4 1/02

(52) U.S. Cl. 546/169

(57) ABSTRACT

Certain compounds of formula (I) below or a pharmaceutically acceptable salt or hydrate thereof:

wherein:

R₁ is H or alkyl;

R₂ is —R₈R₉;

R₈ is a single bond or alkyl, optionally substituted one or more times by hydroxy;

R₉ is aryl or cycloalkyl or heteroaryl, optionally substituted one or more times by hydroxy, alkoxy, or alkoxyalkyl;

R₃ is H or alkyl or cycloalkyl or cycloalkylalkyl, optionally substituted one or more times by hydroxy or by one or more fluorines;

R₄ is —NR₁₀R₁₁;

R₁₀ and R₁₁ are independently selected from H or alkyl, or R₁₀ and R₁₁ together with the nitrogen atom to which they are attached form a saturated or unsaturated heterocyclic ring comprising 3-8 ring members, which heterocyclic ring is unsubstituted or is substituted one or more times by one or more substituents R₁₂;

R₁₂ is oxo or —R₁₃ R₁₄ R₁₅, wherein R₁₃ is a single bond or alkyl, R₁₄ is OC(O) or C(O)O, and R₁₅ is H or alkyl;

R₅ is an alkyl, cycloalkyl, cycloalkylalkyl, aryl, or single or fused ring aromatic heterocyclic group, which group is unsubstituted or is substituted one or more times by one or more substituents selected from halo such as fluoro, alkyl or haloalkyl such as fluoroalkyl;

R₆ represents H or up to three substituents independently selected from the list consisting of: alkyl, alkenyl, aryl, alkoxy or a hydroxylated derivative thereof, hydroxy, halogen, nitro, cyano, carboxy, carboxamido, sulphonamido, alkoxycarbonyl, haloalkyl such as trifluoromethyl, acyloxy, amino, mono- or di-alkylamino, alkoxyamido, alkoxycarboxylate or an esterified derivative thereof;

R₇ is H or halo;

a is 1-6; and

any of R₁, R₃, R₅, R₈, R₉, R₁₀, R₁₁ and R₁₂ may optionally be substituted one or more times by halo, hydroxy, amino, cyano, nitro, carboxy or oxo;

a process for preparing such compounds, a pharmaceutical composition comprising such compounds and the use of such compounds and composition in medicine.

The present invention relates to novel compounds, in particular to novel quinoline derivatives, to processes for the preparation of such compounds, to pharmaceutical compositions containing such compounds and to the use of such compounds in medicine.

The mammalian peptide Neurokinin B (NKB) belongs to the Tachykinin (TK) peptide family which also include Substance P (SP) and Neurokinin A (NKA). Pharmacological and molecular biological evidence has shown the existence of three subtypes of TK receptor (NK₁, NK₂ and NK₃) and NKB binds preferentially to the NK₃ receptor although it also recognises the other two receptors with lower affinity (Maggi et al, 1993, J. Auton. Pharmacol, 13, 23-93).

Selective peptidic NK₃ receptor antagonists are known (Drapeau, 1990 Regul. Pept., 31, 125-135), and findings with peptidic NK₃ receptor agonists suggest that NKB, by activating the NK₃ receptor, has a key role in the modulation of neural input in airways, skin, spinal cord and nigro-striatal pathways (Myers and Undem, 1993, J. Physiol., 470, 665-679; Counture et al., 1993, Regul. Peptides, 46, 426-429; Mccarson and Krause, 1994, J. Neurosci., 14 (2), 712-720; Arenas et al. 1991, J. Neurosci., 11, 2332-8). However, the peptide-like nature of the known antagonists makes them likely to be too labile from a metabolic point of view to serve as practical therapeutic agents.

International Patent Application, Publication number WO 00/31037 discloses certain compounds stated to be non-peptide NK-3 antagonists and also to have NK-2 antagonist activity. These compounds are disclosed to be of potential use in the prevention and treatment of a wide variety of clinical conditions, which are characterised by overstimulation of the Tachykinin receptors, in particular NK-3 and NK-2.

We have now discovered a further novel class of potent non-peptide NK-3 antagonists some of which fall within the generic scope of WO 00/31037. The new compounds are also far more stable from a metabolic point of view than the known peptidic NK-3 receptor antagonists and are of potential therapeutic utility. The new compounds also have good NK-2 antagonist activity and are therefore considered to be of potential use in the prevention and treatment of a wide variety of clinical conditions which are characterised by overstimulation of the Tachykinin receptors, in particular NK-3 and NK-2.

These conditions include respiratory diseases, such as chronic obstructive pulmonary disease (COPD), asthma, airway hyper-reactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (TBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies (hereinafter referred to as the ‘Primary Conditions’).

Certain of these compounds also show CNS activity and hence are considered to be of particular use in the treatment of disorders of the central nervous system such as anxiety, depression, psychosis and schizophrenia; neurodegenerative disorders such as AIDS related dementia, senile dementia of the Alzheimer type, Alzheimer's disease, Down's syndrome, Huntingdon's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilatation and vasospastic diseases such as angina, migraine and Reynaud's disease and pain or nociception, for example, that is attributable to or associated with any of the foregoing conditions especially the transmission of pain in migraine, (hereinafter referred to as the ‘Secondary Conditions’).

The new compounds also show improved oral bioavailability.

The compounds of formula (I) are also considered to be useful as diagnostic tools for assessing the degree to which neurokinin-3 and neurokinin-2 receptor activity (normal, overactivity or underactivity) is implicated in a patient's symptoms.

According to the present invention, there is provided a compound of formula (I) below or a pharmaceutically acceptable salt or hydrate thereof:

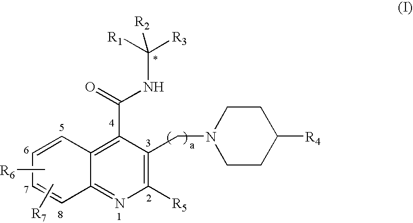

wherein:

R₁ is H or alkyl;

R₂ is —R₈R₉;

R₈ is a single bond or alkyl, optionally substituted one or more times by hydroxy;

R₉ is aryl or cycloalkyl or heteroaryl, optionally substituted one or more times by hydroxy, alkoxy, or alkoxyalkyl;

R₃ is H or alkyl or cycloalkyl or cycloalkylalkyl, optionally substituted one or more times by hydroxy or by one or more fluorines,

R₄ is —NR₁₀R₁₁;

R₁₀ and R₁₁ are independently selected from H or alkyl, or R₁₀ and R₁₁ together with the nitrogen atom to which they are attached form a saturated or unsaturated heterocyclic ring comprising 3-8 ring members, which heterocyclic ring is unsubstituted or is substituted one or more times by one or more substituents R₁₂;

R₁₂ is oxo or —R₁₃ R₁₄ R₁₅, wherein R₁₃ is a single bond or alkyl, R₁₄ is OC(O) or C(O)O, and R₁₅ is H or alkyl;

R₅ is an alkyl, cycloalkyl, cycloalkylalkyl, aryl, or single or fused ring aromatic heterocyclic group, which group is unsubstituted or is substituted one or more times by one or more substituents selected from halo such as fluoro, alkyl or haloalkyl such as fluoroalkyl;

R₆ represents H or up to three substituents independently selected from the list consisting of: alkyl, alkenyl, aryl, alkoxy or a hydroxylated derivative thereof, hydroxy, halogen, nitro, cyano, carboxy, carboxamido, sulphonamido, alkoxycarbonyl, haloalkyl such as trifluoromethyl, acyloxy, amino, mono- or di-alkylamino, alkoxyamido, alkoxycarboxylate or an esterified derivative thereof;

R₇ is H or halo;

a is 1-6; and

any of R₁, R₃, R₅, R₈, R₉, R₁₀, R₁₁ and R₁₂ may optionally be substituted one or more times by halo, hydroxy, amino, cyano, nitro, carboxy or oxo;

with the proviso that the compound is not a compound in which R₇ represents H, R₅ represents unsubstituted phenyl, and R₁, R₂, R₃, R₄, R₆ and a are selected from one of the following combinations:

| 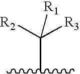 | R₆ | 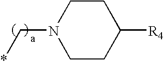 |
|---|---|---|
| 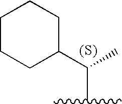 | H | 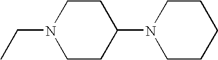 |
| 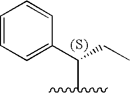 | H | 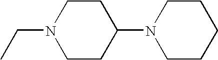 |
| 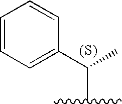 | H | 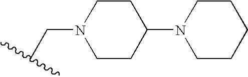 |
| 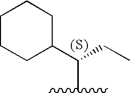 | H | 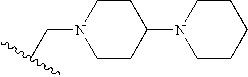 |
| 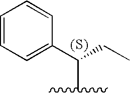 | H | 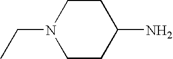 |
| 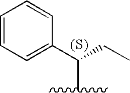 | 7-OMe, 8-Br | 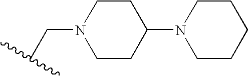 |
| 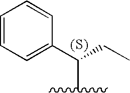 | 7-OMe | 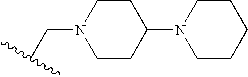 |
| 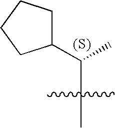 | H | 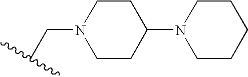 |
| 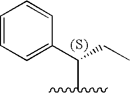 | H | 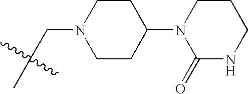 |
| 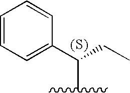 | H | 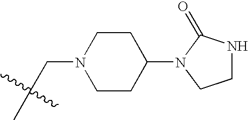 |
| 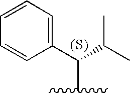 | H | 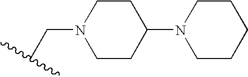 |
| 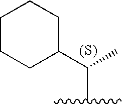 | 7-OMe | 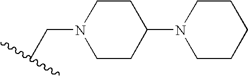 |
| 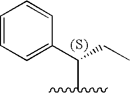 | 7-OH, 8-Cl | 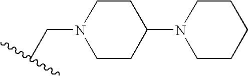 |
| 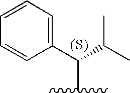 | H | 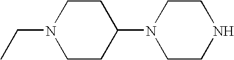 |
| 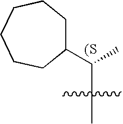 | H | 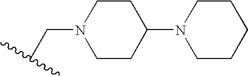 |
| 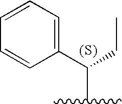 | 7-OH | 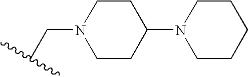 |
| 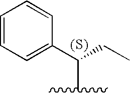 | H | 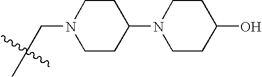 |
| 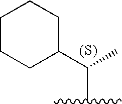 | H | 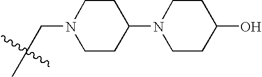 |
| 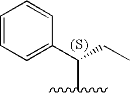 | H | 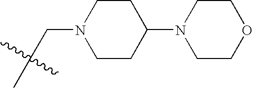 |
| 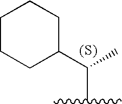 | H | 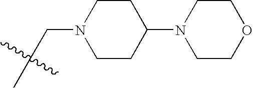 |

Advantageously, R₃ may represent methyl, ethyl, iso-propyl, cyclopropyl, hydroxymethyl or hydroxyethyl.

Suitably, R₈ may represent a single bond.

Alternatively, R₈ may represent hydroxymethyl.

Advantageously, R₉ may represent phenyl or cyclohexyl, which phenyl or cyclohexyl may be unsubstituted or may be substituted, preferably para-substituted, by hydroxy or alkoxy such as methoxy or alkoxyalkyl such as methoxymethyl, methoxyethyl, methoxypropyl or methoxybutyl.

In preferred embodiments, R₁ is hydrogen.

Suitably, R₅ may be unsubstituted phenyl. Alternatively, R₅ may be phenyl which is substituted one or more times by halo such as fluoro, and/or haloalkyl such as trifluoromethyl. Preferably, said phenyl may be ortho- or para-substituted by said halo, or may be para-substituted by said haloalkyl. As yet a further alternative, R₅ may be a heterocyclic ring, such as an unsaturated heterocyclic ring, comprising at least one heteroatom such as S. In particular, R₅ may be

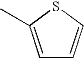

Preferably, R₇ may represent hydrogen.

In some embodiments, R₆ represents hydrogen, or one or more substituents selected from fluoro, chloro, bromo or trifluoromethyl. Said one or more substituents may preferably be positioned at the 5′, 6′, 7′ and/or 8′ positions around the quinoline ring of the compound of formula (I). More preferably, said one or more substituents may preferably be positioned at the 6′ and/or 7′ positions around the quinoline ring of the compound of formula (I). Advantageously, said one or more substituents may comprise a trifluoromethyl group which is positioned at the 6′ or the 7′ position around the quinoline ring. Alternatively, said one or more substituents may comprise a fluorine group which is positioned at the 5′, 6′ or 7′ position around said quinoline ring.

In other embodiments, R₆ represents one ring substituent, which is hydroxy, alkoxy such as methoxy or ethoxy or a hydroxylated derivative thereof, alkoxycarboxylate such as methoxycarboxylate or ethoxycarboxylate or an esterified derivative thereof such as methoxyethanoate ethoxyethanoate, or alkoxyamido such as methoxyamido or ethoxyamido. Said one ring substituent may be located at the 6 or 7 position around said quinoline ring.

Advantageously, a may be 1, 2 or 3.

In some embodiments, each of R₁₀ and R₁₁ is hydrogen.

In other embodiments, R¹⁰ and R₁₁ together with the nitrogen atom to which they are attached form a saturated heterocyclic ring comprising five or six ring members. Said saturated heterocyclic ring may comprise one or more additional nitrogen atoms. Optionally, said saturated heterocyclic ring may be substituted by oxo. Said saturated heterocyclic ring may additionally or alternatively be substituted by —R₁₃ R₁₄ R₁₅, wherein R₁₃ is methyl, ethyl, propyl or butyl, and R₁₅ is H or methyl, ethyl, propyl or butyl. Suitably, R₁₄ is C(O)O.

In especially preferred embodiments, R₅ is unsubstituted phenyl, R₆ is H, R₇ is H, and a, R₁, R₂, R₃, and R are selected from the following combinations:

| 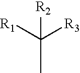 | 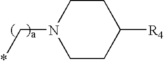 |
|---|---|
| 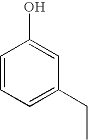 | 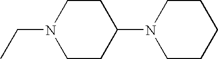 |
| 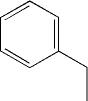 | 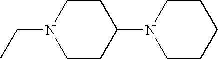 |
| 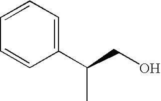 | 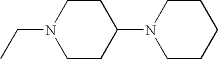 |
| 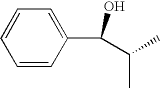 | 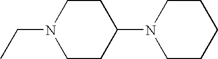 |
| 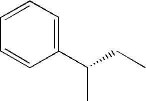 | 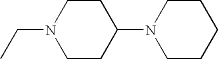 |
| 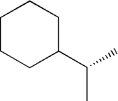 | 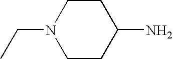 |
| 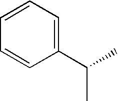 | 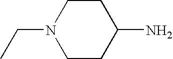 |
| 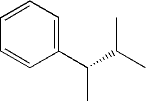 | 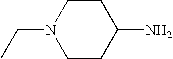 |
| 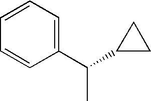 | 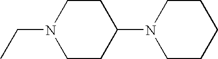 |
| 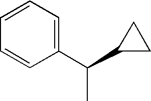 | 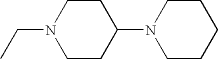 |
| 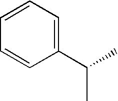 | 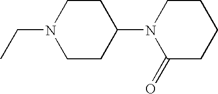 |
| 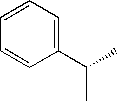 | 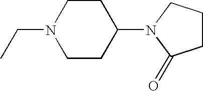 |
| 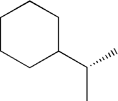 | 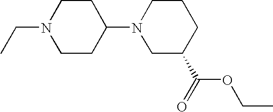 |
| 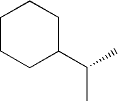 | 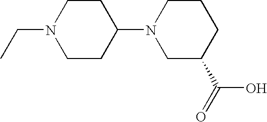 |
| 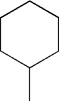 | 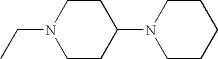 |

In other especially preferred embodiments, the compound of the present invention is selected from the following:

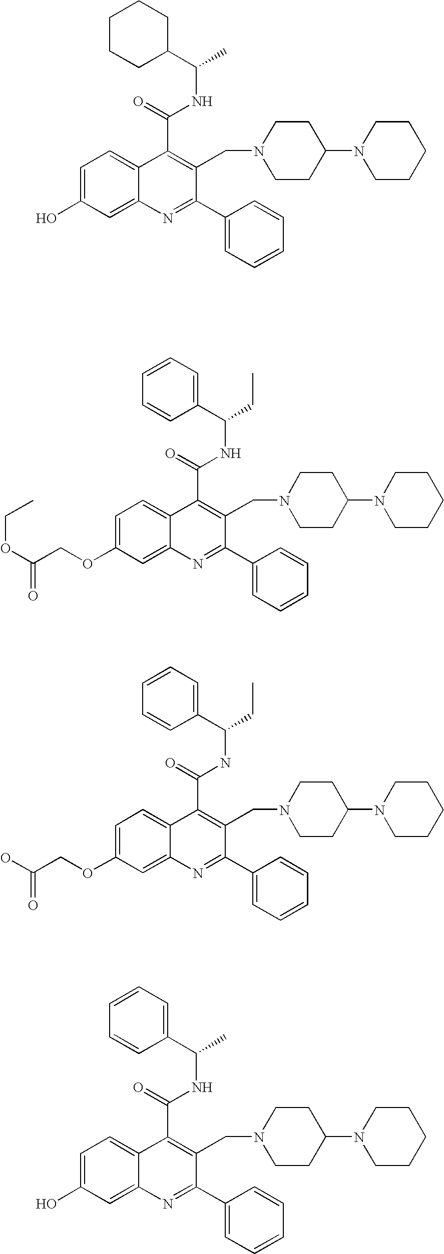

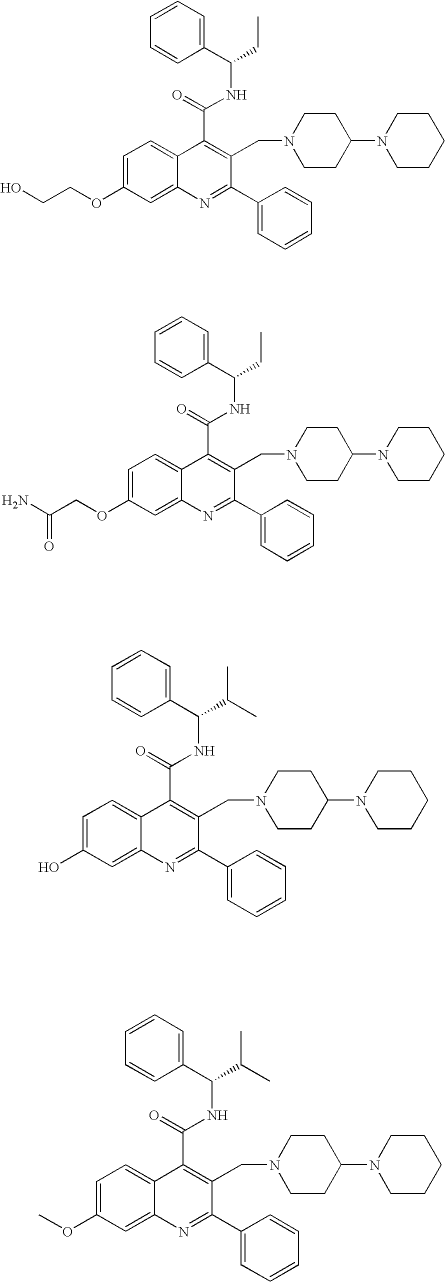

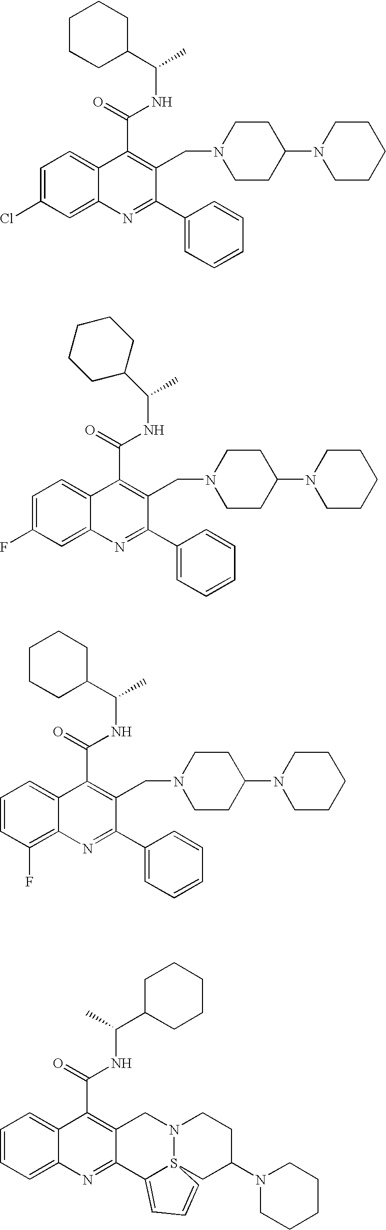

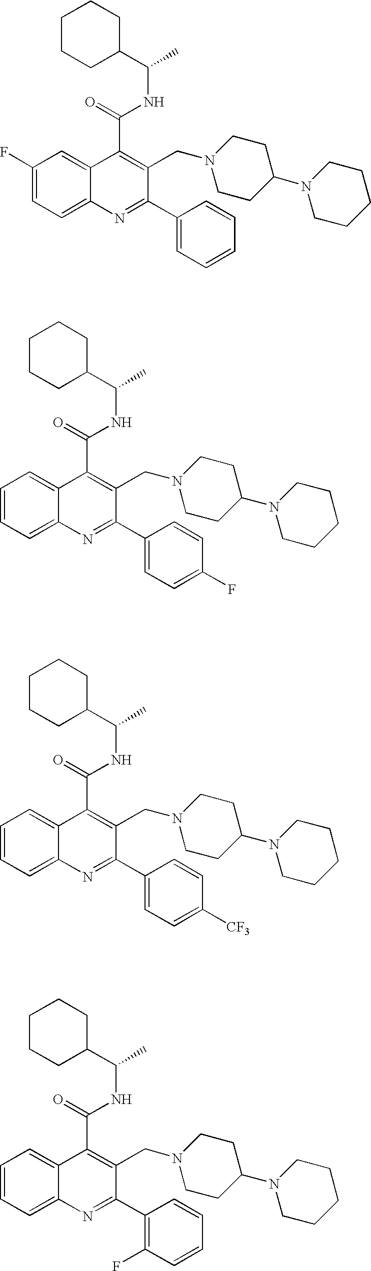

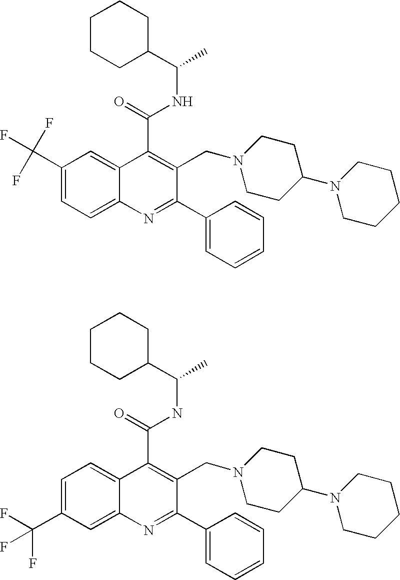

More particularly, the compound of the present invention may be selected from the following:

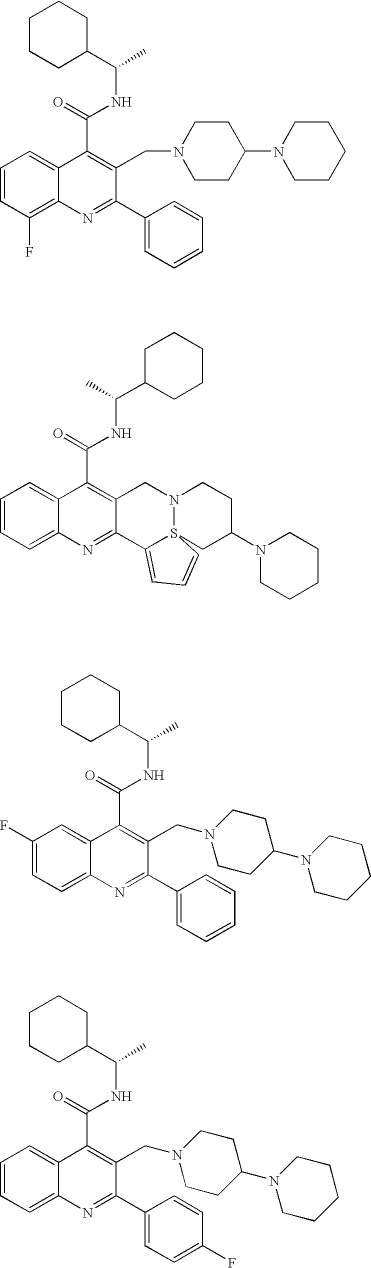

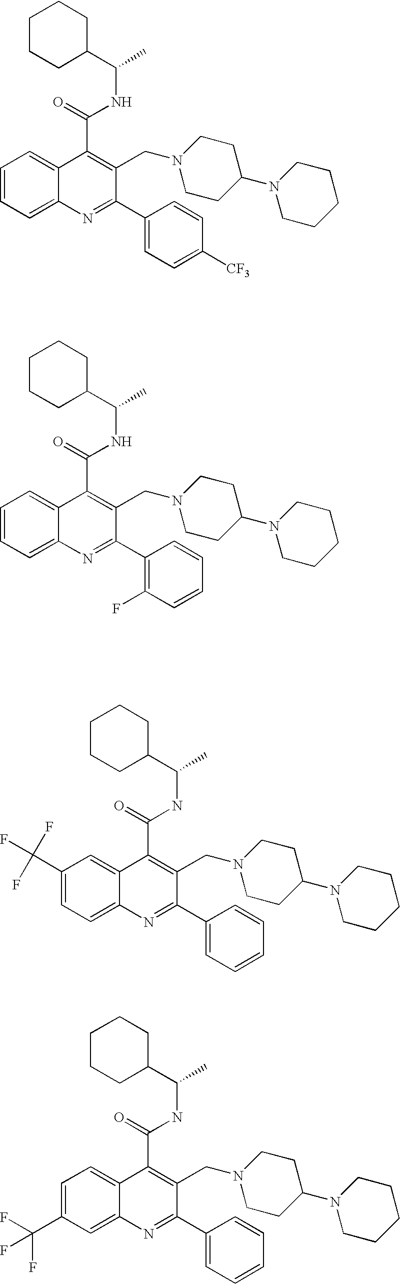

The compounds of formula (I) may have at least one asymmetric centre—for example the carbon atom labelled with an asterisk (*) in the compound of formula (I)—and therefore may exist in more than one stereoisomeric form. The invention extends to all such stereoisomeric forms and to mixtures thereof, including racemates. In particular, the invention includes compounds wherein the asterisked carbon atom in formula (I) has the stereochemistry shown in formula (Ia):

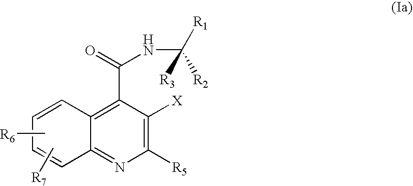

wherein R₁, R₂, R₃, R₅, R₆, and R₇ are as defined in relation to formula (I), and X represents the moiety

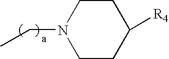

The compounds of formula (_) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels.

A substantially pure form will generally contain at least 50% (excluding normal pharmaceutical additives), preferably 75%, more preferably 90% and still more preferably 95% of the compound of formula (I) or its salt or solvate.

One preferred pharmaceutically acceptable form is the crystalline form, including such form in pharmaceutical composition. In the case of salts and solvates the additional ionic and solvent moieties must also be non-toxic.

Suitable salts are pharmaceutically acceptable salts.

Suitable pharmaceutically acceptable salts include the acid addition salts with the conventional pharmaceutical acids, for example maleic, hydrochloric, hydrobromic, phosphoric, acetic, fumaric, salicylic, citric, lactic, mandelic, tartaric, succinic, benzoic, ascorbic and methanesulphonic.

Suitable pharmaceutically acceptable salts include salts of acidic moieties of the compounds of formula (I) when they are present, for example salts of carboxy groups or phenolic hydroxy groups.

Suitable salts of acidic moieties include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-β-phenethylarnie, dehydroabietylamine, N,N′-bisdehydroabietylamine, glucamine, N-methylglucamine or bases of the pyridine type such as pyridine, collidine, quinine or quinoline.

Suitable solvates are pharmaceutically acceptable solvates.

Suitable pharmaceutically acceptable solvates include hydrates.

The term ‘alkyl’ (unless specified to the contrary) when used alone or when forming part of other groups (such as the ‘alkoxy’ group) denotes straight- or branched-chain alkyl groups containing 1 to 12 carbon atoms, suitably 1 to 6 carbon atoms, examples include methyl, ethyl, n-propyl, isopropyl, n-butyl, isobutyl or tert-butyl group.

The term ‘carbocylic’ denotes cycloalkyl and aryl rings.

The term ‘cycloalkyl’ denotes groups having 3 to 12, suitably 4 to 6 ring carbon atoms.

The term ‘aryl’ denotes aromatic groups including phenyl and naphthyl, preferably phenyl which unless specified to the contrary optionally comprise up to five, preferably up to three substituents selected from halogen, alkyl, phenyl, alkoxy, haloalkyl, hydroxyalkyl, hydroxy, amino, nitro, cyano, carboxy, alkoxycarbonyl, alkoxycarbonylalkyl, alkylcarbonyloxy, or alkylcarbonyl groups.

The term ‘aromatic heterocyclic group’ denotes groups comprising aromatic heterocyclic rings containing from 5 to 12 ring atoms, suitably 5 or 6, and comprising up to four hetero-atoms in the or each ring selected from S, O or N.

Unless specified to the contrary, suitable substituents for any heterocyclic group includes up to 4 substituents selected from the group consisting of: alkyl, alkoxy, aryl and halogen or any two substituents on adjacent carbon atoms, together with the carbon atoms to which they are attached, may form an aryl group, preferably a benzene ring, and wherein the carbon atoms of the aryl group represented by the said two substituents may themselves be substituted or unsubstituted.

When used herein the term “halogen” refers to fluorine, chlorine, bromine and iodine, preferably fluorine, chlorine or bromine.

It will be understood that unless specified to the contrary, groups and substituents specified herein are unsubstituted.

When used herein the term “acyl” includes residues of acids, in particular a residue of a carboxylic acid such as an alkyl- or aryl-carbonyl group.

The invention also provides a process for the preparation of a compound of formula (I), or a salt thereof and/or a solvate thereof, which process comprises reacting a compound of formula (II) or an active derivative thereof:

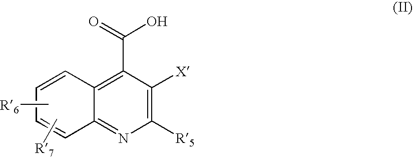

wherein R′₆, R′₇, R′₅ and X′ are R₆, R₇, R₅ and X respectively as hereinbefore defined in relation to formula (I) or (Ia), or a group convertible to R₆, R₇, R₅ and X respectively; with a compound of formula (III):

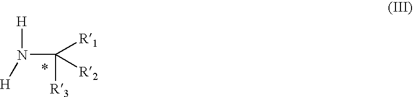

wherein R₁′, R₂′, and R₃′ are R₁, R₂, and R₃ as defined for formula (I) or a group or atom convertible to R₁, R₂, and R₃ respectively; to form a compound of formula (Ib):

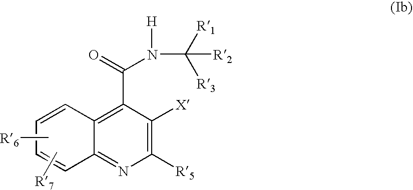

wherein R′₁, R′₂, R′₃, X′, R′₅, R′₆ and R′₇ are as defined above, and thereafter carrying out one or more of the following optional steps:

(i) converting any one of R′₁, R′₂, R′₃, X′, R′₅, R′₆ and R′₇ to R₁, R₂, R₃, X, R₅, R₆ and R₇ respectively as required, to obtain a compound of formula (I);

(ii) converting a compound of formula (I) into another compound of formula (I); and

(iii) preparing a salt of the compound of formula (I) and/or a solvate thereof.

Suitable groups convertible into other groups include protected forms of said groups.

Suitably R′₁, R′₂, R′₃, X′, R′₅, R′₆ and R′₇ each represents R₁, R₂, R₃, X, R₅, R₆ and R₇ respectively or a protected form thereof.

It is favoured if the compound of formula (II) is present as an active derivative.

A suitable active derivative of a compound of formula (II) is a transient activated form of the compound of formula (II) or a derivative wherein the carboxy group of the compound of formula (II) has been replaced by a different group or atom, for example by an acyl halide, preferably a chloride, or an acylazide or a carboxylic acid anhydride.

Other suitable active derivatives include: a mixed anhydride formed between the carboxyl moiety of the compound of formula (II) and an alkyl chloroformate; an activated ester, such as a cyanomethyl ester, thiophenyl ester, p-nitrophenyl ester, p-nitrothiophenyl ester, 2,4,6-trichlorophenyl ester, pentachlorophenyl ester, pentafluorophenyl ester, N-hydroxy-phtalimido ester, N-hydroxypiperidine ester, N-hydroxysuccinimide ester, N-hydroxy benzotriazole ester; alternatively, the carboxy group of the compound of formula (II) may be activated using a carbodiimide or N,N′-carbonyldiimidazole.

The reaction between the compound of formula (II) or the active derivative thereof and the compound of formula (III) is carried out under the appropriate conventional conditions for the particular compounds chosen. Generally, when the compound of formula (II) is present as an active derivative the reaction is carried out using the same solvent and conditions as used to prepare the active derivative, preferably the active derivative is prepared in situ prior to forming the compound of formula (Ib) and thereafter the compound of formula (I) or a salt thereof and/or a solvate thereof is prepared.

For example, the reaction between an active derivative of the compound of formula (II) and the compound of formula (III) may be carried out:

(a) by first preparing an acid chloride and then coupling said chloride with the compound of formula (III) in the presence of an inorganic or organic base in a suitable aprotic solvent such as dimethylformamide (DMF) at a temperature in a range from −70 to 50° C. (preferably in a range from −10 to 20° C.); or

(b) by treating the compound of formula (II) with a compound of formula (III) in the presence of a suitable condensing agent, such as for example N,N′-carbonyl diimidazole (CDI) or a carbodiimide such as dicyclohexylcarbodiimide (DCC) or N-dimethylaminopropyl-N′-ethylcarbodiimide, preferably in the presence of N-hydroxybenzotriazole (HOBT) to maximise yields and avoid racemization processes (see Synthesis, 453, 1972), or O-benzotriazol-1-yl-N,N,N′,N′-tetramethyluroniumhexafluorophosphate (HBTU), in an aprotic solvent, such as a mixture of acetonitrile (MeCN) and tetrahydrofuran (THF), for example a mixture in a volume ratio of from 1:9 to 7:3 (MeCN:THF), at any temperature providing a suitable rate of formation of the required product, such as a temperature in the range of from −70 to 50° C., preferably in a range of from −10 to 25° C., for example at 0° C.

A preferred reaction is set out in Scheme 1 shown below:

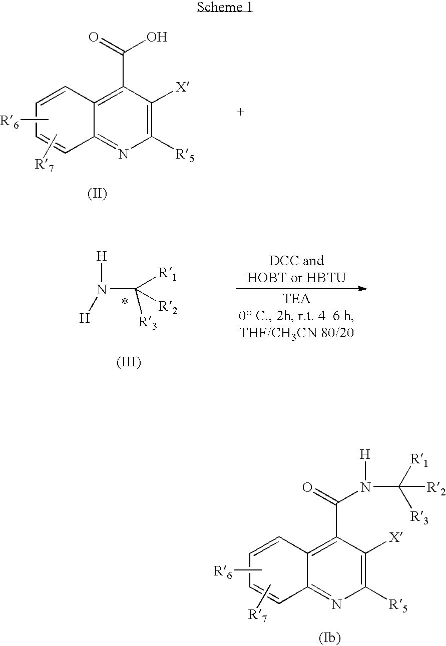

wherein R′₁, R′₂, R′₃, X′, R′₅, R′₆ and R′₇ are as defined above.

It will be appreciated that a compound of formula (Ib) may be converted to a compound of formula (I), or one compound of formula (I) may be converted to another compound of formula (I) by interconversion of suitable substituents. Thus, certain compounds of formula (I) and (Ib) are useful intermediates in forming other compounds of the present invention.

Accordingly, in a further aspect the invention provides a process for preparing a compound of formula (I), or a salt thereof and/or a solvate thereof, which process comprises converting a compound of the above defined formula (Ib) wherein at least one of R′₁, R′₂, R′₃, X′, R′₅, R′₆ and R′₇ is not R₁, R₂, R₃, X, R₅, R₆ or R₇ respectively, thereby to provide a compound of formula (I); and thereafter, as required, carrying out one or more of the following optional steps:

(i) converting a compound of formula (I) into another compound of formula (I); and

(ii) preparing a salt of the compound of formula (I) and/or a solvate thereof.

Suitably, in the compound of formula (Ib) the variables R′₁, R′₂, R′₃, X′, R′₅, R′₆ and R′₇ are R₁, R₂, R₃, X, R₅, R₆ and R₇ respectively or they are protected forms thereof.

The above mentioned conversions, protections and deprotections are carried out using the appropriate conventional reagents and conditions and are further discussed below.

A compound of formula (II) or the corresponding alkyl (such as methyl or ethyl) ester, is prepared by reacting a compound of formula (IV) or the corresponding alkyl (such as methyl or ethyl) ester:

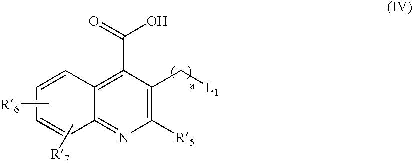

wherein R′₆, R′₇, R′₅ and a are as defined above and L₁ represents a halogen atom such as a bromine atom, with a compound of formula (V):

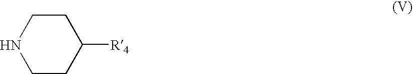

wherein R′₄ is R₄ as defined in relation to formula (I) or a protected form thereof.

Suitably, R′₄ is R₄.

Suitably, reaction between the compounds of formulae (IV) or the corresponding alkyl (such as methyl or ethyl) ester and (V) is carried out under conventional amination conditions, for example when L₁ is a bromine atom then the reaction is conveniently carried out in an aprotic solvent, such as tetrahydrofaran or dimethylformamide at any temperature providing a suitable rate of formation of the required product, usually at ambient temperature; preferably the reaction is carried out in the presence of triethylamine (TEA) or K₂CO₃.

A compound of formula (IV) or the corresponding alkyl (such as methyl or ethyl) ester is prepared by appropriate halogenation of a compound of formula (VI) or the corresponding alkyl (such as methyl or ethyl) ester:

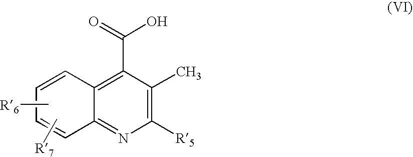

wherein R′₆, R′₇ and R′₅ are as defined above in relation to formula (II).

Suitable halogenation reagents are conventional reagents depending upon the nature of the halogen atom required, for example when L₁ is bromine a preferred halogenation reagent is N-bromosuccinimide (NBS).

The halogenation of the compound of formula (VI) or the corresponding alkyl (such as methyl or ethyl) ester is carried out under conventional conditions, for example bromination is carried out by treatment with NBS in an inert solvent, such as carbon tetrachloride CCl₄, or 1,2-dichloroethane or CH₃CN, at any temperature providing a suitable rate of formation of the required product, suitably at an elevated temperature such as a temperature in the range of 60° C. to 100° C., for example 80° C.; preferably the reaction is carried out in the presence of a catalytic amount of benzoyl peroxide.

In the case in which the corresponding alkyl (such as methyl or ethyl) ester of compounds (VI), (IV) and (II) are utilised, an hydrolysis to compound (II) is required before conversion to compound (Ib) in Scheme 1. Such hydrolysis can be carried out under acidic conditions, such 10-36% hydrochloric acid at a temperature in the range between 30 and 100° C. A compound of formula (II) wherein X′ represents

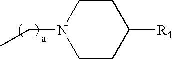

is conveniently prepared by reacting a compound of formula (VII):

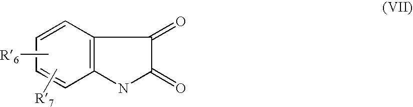

wherein R′₆ and R′₇ are as defined in relation to formula (II), with a compound of formula (VIII):

R₅′—CO—CH₂—(CH₂)a-T₅ (VIII)

wherein R′₅ is as defined in relation to formula (II), and T₅ is a group

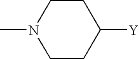

where Y is a protecting group such as a benzyl group, particularly a protecting group which is stable in basic conditions such as a terbutoxycarbonyl group, or a group R₄ as defined in relation to formula (I) or a protected form thereof or a group convertible thereto, and a is an integer in the range of 1 to 6; and thereafter as required removing any protecting group, for example by dehydrogenation, and/or converting any group T₅ to

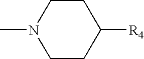

The reaction between the compounds of formula (VII) and (VIII) is conveniently carried out using Pfitzinger reaction conditions (see for example J. Prakt. Chem. 33, 100 (1886), J. Prakt. Chem. 38, 582 (1888), J. Chem. Soc. 106 (1948) and Chem. Rev. 35, 152 (1944)), for example in an alkanolic solvent such as ethanol, at any temperature providing a suitable rate of formation of the required product, but generally at an elevated temperature, such as the reflux temperature of the solvent, and preferably in the presence of a base such as potassium hydroxide or potassium tert-butoxide.

Protected forms of

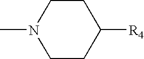

will vary according to the particular nature of the group being protected but will be chosen in accordance with normal chemical practice.

Groups convertible to

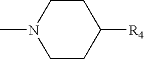

include groups dictated by conventional chemical practice to be required and to be appropriate, depending upon the specific nature of the

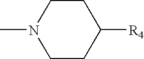

under consideration.

Suitable deprotection methods for deprotecting protected forms of

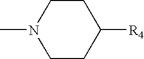

and conversion methods for converting T₅ to

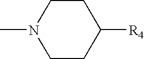

will be those used conventionally in the art depending upon the particular groups under consideration with reference to standard texts such as Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994 and Chemistry of the Amino Group, Patais (Ed.), Interscience, New York 1968; or Advanced Organic Chemistry, March J, John Wiley & Sons, New York, 1992.

A compound of formula (VIII) is prepared from a compound of formula (IX):

R₅′—CO—CH₂—(CH₂)ₐ—OH (IX)

wherein R′₅ is as defined in relation to formula (II) and a is as defined in relation to formula (VIII), by first halogenating, preferably brominating, or mesylating the compound of formula (IX) and thereafter reacting the halogenation or mesylation product so formed with a compound capable of forming a group T₅ so as to provide the required compound of formula (VII).

When T₅ is a group

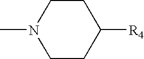

a compound capable of forming a group T₅ is a compound of the above defined formula (V).

The halogenation of the compound of formula (IX) is suitably carried out using a conventional halogenation reagent. Mesylation is conveniently carried out using mesyl chloride in an inert solvent such as methylene dichloride, at a temperature below room temperature, such as 0° C., preferably in the presence of triethylamine. The reaction conditions between the compound of formula (IX) and the compound capable of forming a group T₅ will be those conventional conditions dictated by the specific nature of the reactants, for example when the T₅ required is a group

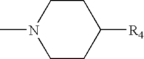

and the required compound capable of forming a group T₅ is a compound of the above defined formula (V), then the reaction between the halogenation or mesylation product of the compound of formula (IX) and the compound of formula (V) is carried out under analogous conditions to those described for the reaction between the compounds of formulae (IV) and (V).

Other compounds capable of forming a group T₅ will depend upon the particular nature of T₅, but will be those appropriate compounds dictated by conventional chemical practice with reference to standard texts such as Chemistry of the Amino Group, Patais (Ed.), Interscience, New York 1968; and Advanced Organic Chemistry, March J, John Wiley & Sons, New York, 1992.

A compound of formula (IX) may be prepared by reacting a compound of formula (X):

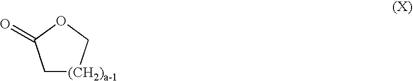

wherein a is as defined in relation to formula (VIII), with a lithium salt of formula (XI):

R′₅Li (XI)

wherein R′₅ is as defined in relation to formula (I).

The reaction between the compounds of formulae (X) and (XI) can be carried out in an aprotic solvent, such as diethyl-ether at any temperature providing a suitable rate of formation of the required product, usually at a low temperature such as in the range of −10° C. to −30° C., for example −20° C.

The compounds of formula (III) are known commercially available compounds or they can be prepared from known compounds by known methods, or methods analogous to those used to prepare known compounds, for example the methods described in Liebigs Ann. der Chemie, (1936), 523, 199.

A chiral compound of formula (m) wherein R₂ is a C₅ or C₇ cycloalkyl group, R₃ is methyl and R₁ is H are described in J. Org. Chem. (1996), 61 (12), 4130-4135. A chiral compound of formula (III) wherein R₂ is phenyl, R₃ is isopropyl and R₁ is H is a known compound described in for example Tetrahedron Lett. (1994), 35(22), 3745-6.

The compounds of formula (V) are known, commercially available compounds or they can be prepared using methods analogous to those used to prepare known compounds; for example the methods described in the Chemistry of the Amino Group, Patais (Ed.), Interscience, New York 1968; Advanced Organic Chemistry, March J, John Wiley & Sons, New York, 1992; J. Heterocyclic Chem. (1990), 27, 1559; Synthesis (1975), 135, Bioorg. Med. Chem. Lett. (1997), 7, 555, or Protective Groups in Organic Synthesis (second edition), Wiley Interscience, (1991) or other methods mentioned herein.

The compounds of formula (VII) are known compounds or they are prepared according to methods used to prepare known compounds for example those disclosed in J. Org. Chem. 21, 171 (1955); J. Org. Chem. 21, 169 (1955).

The compounds of formula (X) and (XI) are known compounds or they are prepared according to methods used to prepare known compounds for example those disclosed by Krow G. R. in Organic Reactions, Vol 43, page 251, John Wiley & Sons Inc. 1994 (for the compounds of formula (X)) and Organometallics in Synthesis, Schlosser M. (Ed), John Wiley & Sons Inc. 1994 (for the compounds of formula (XI)).

As hereinbefore mentioned, the compounds of formula (I) may exist in more than one stereoisomeric form—and the process of the invention may produce racemates as well as enantiomerically pure forms. Accordingly, a pure enantiomer of a compound of formula (I) is obtained by reacting a compound of the above defined formula (II) with an appropriate enantiomerically pure primary amine of formula (IIIa) or (IIIc):

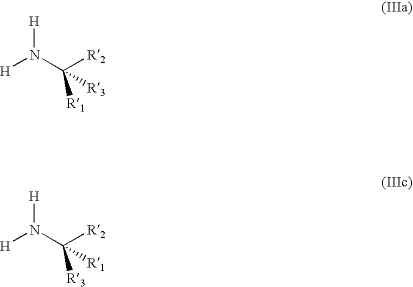

wherein R′₁, R′₂ and R′₃ are as defined above, to obtain a compound of formula (I′a) or (I′c):

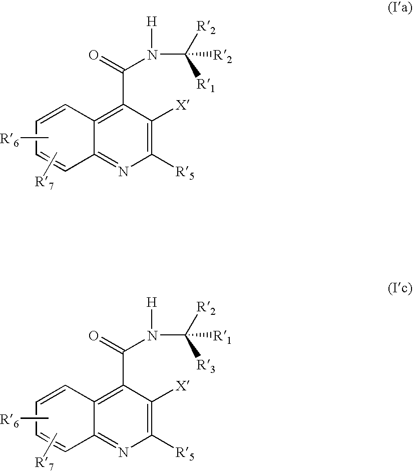

wherein R′₁, R′₂, R′₃, X′, R′₅, R′₆, and R′₇ are as defined above.

Compounds of formula (I′a) or (I′c) may subsequently be converted to compounds of formula (I′a) or (I′c) by the methods of conversion mentioned before:

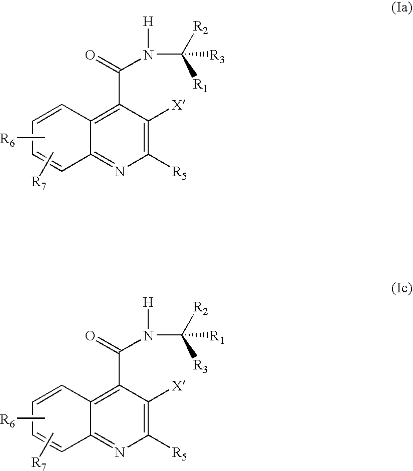

wherein R₁, R₂, R₃, X, R₅, R⁶, and R₇ are as defined above.

Suitably, in the above mentioned compounds of formulae (Ia), (Ic), (I′a), (I′c), (IIIa) and (IIIc) R₁ represents hydrogen.

An alternative method for separating optical isomers is to use conventional, fractional separation methods in particular fractional crystallization methods. Thus, a pure enantiomer of a compound of formula (I) is obtained by fractional crystallisation of a diastereomeric salt formed by reaction of the racemic compound of formula (I) with an optically active strong acid resolving agent, such as camphosulphonic acid, tartaric acid, O,O′-di-p-toluoyltartaric acid or mandelic acid, in an appropriate alcoholic solvent, such as ethanol or methanol, or in a ketonic solvent, such as acetone. The salt formation process should be conducted at a temperature between 20° C. and 80° C., preferably at 50° C.

A suitable conversion of one compound of formula (I) into a further compound of formula (I) involves converting one group X into another group X by for example:

(i) converting a ketal into a ketone, by such as mild acidic hydrolysis, using for example dilute hydrochloric acid;

(ii) reducing a ketone to a hydroxyl group by use of a borohydride reducing agent;

(iii) converting a carboxylic ester group into a carboxyl group using basic hydrolysis; and/or

(iv) reducing a carboxylic ester group to a hydroxymethyl group, by use of a borohydride reducing agent.

As indicated above, where necessary, the conversion of any group R′₁, R′₂, R′₃, X′, R′₅, R′₆, and R′₇ into R₁, R₂, R₃, X, R₅, R₆, and R₇ which as stated above are usually protected forms of R₁, R₂, R₃, X, R₅, R₆, or R₇ may be carried out using appropriate conventional conditions such as the appropriate deprotection procedure.

It will be appreciated that in any of the above mentioned reactions any reactive group in the substrate molecule may be protected and deprotected according to conventional chemical practice, for example as described by Greene, T. W. and Wuts, P. G. M. Protective Groups in Organic Synthesis, John Wiley & Sons Inc. New York, 1991 (Second Edt.) or in Kocienski, P. J. Protecting groups. George Thieme Verlag, New York, 1994.

Suitable protecting groups in any of the above mentioned reactions are those used conventionally in the art. Thus, for example suitable hydroxyl protecting groups include benzyl or trialkylsilyl groups.

The methods of formation and removal of such protecting groups are those conventional methods appropriate to the molecule being protected. Thus for example a benzyloxy group may be prepared by treatment of the appropriate compound with a benzyl halide, such as benzyl bromide, and thereafter, if required, the benzyl group may be conveniently removed using catalytic hydrogenation or a mild ether cleavage reagent such as trimethylsilyl iodide or boron tribromide.

As indicated above, the compounds of formula (I) have useful pharmaceutical properties.

Accordingly the present invention also provides a compound of formula (I), or a pharmaceutically acceptable salt or solvate thereof, for use as an active therapeutic substance.

In particular, the present invention also provides a compound of formula (I), or a pharmaceutically acceptable salt or solvate thereof, for the treatment or prophylaxis of the Primary and Secondary Conditions.

The present invention further provides a pharmaceutical composition comprising a compound of formula (I), or a pharmaceutically acceptable salt or solvate thereof, and a pharmaceutically acceptable carrier.

The present invention also provides the use of a compound of formula (I), or a pharmaceutically acceptable salt or solvate thereof, in the manufacture of a medicament for the treatment of the Primary and Secondary Conditions.

As mentioned above the Primary conditions include respiratory diseases, such as chronic obstructive pulmonary disease (COPD), asthma, airway hyperreactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation, conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease, irritable bowel syndrome (IBS), gastro-exophageous reflex disease (GERD); urinary incontinence and disorders of the bladder function; renal disorders; increased blood pressure, proteinuria, coagulopathy and peripheral and cerebral oedema following pre-eclampsia in pregnancies.

As mentioned above, the Secondary conditions include disorders of the central nervous system such as anxiety, depression, psychosis and schizophrenia; neurodegenerative disorders such as AIDS related dementia, senile dementia of the Alzheimer type, Alzheimer's disease, Down's syndrome, Huntington's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilation and vasospastic diseases such as angina, migraine and Reynaud's disease and pain or nociception, for example, that is attributable to or associated with any of the foregoing conditions especially the transmission of pain in migraine.

Such a medicament, and a composition of this invention, may be prepared by admixture of a compound of the invention with an appropriate carrier. It may contain a diluent, binder, filler, disintegrant, flavouring agent, colouring agent, lubricant or preservative in conventional manner.

These conventional excipients may be employed for example as in the preparation of compositions of known agents for treating the conditions.

Preferably, a pharmaceutical composition of the invention is in unit dosage form and in a form adapted for use in the medical or veterinarial fields. For example, such preparations may be in a pack form accompanied by written or printed instructions for use as an agent in the treatment of the conditions.

The suitable dosage range for the compounds of the invention depends on the compound to be employed and on the condition of the patient. It will also depend, inter alia, upon the relation of potency to absorbability and the frequency and route of administration.

The compound or composition of the invention may be formulated for administration by any route, and is preferably in unit dosage form or in a form that a human patient may administer to himself in a single dosage. Advantageously, the composition is suitable for oral, rectal, topical, parenteral, intravenous or intramuscular administration. Preparations may be designed to give slow release of the active ingredient.

Compositions may, for example, be in the form of tablets, capsules, sachets, vials, powders, granules, lozenges, reconstitutable powders, or liquid preparations, for example solutions or suspensions, or suppositories.

The compositions, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinyl-pyrrolidone, sodium starch glycollate or microcrystalline cellulose; or pharmaceutically acceptable setting agents such as sodium lauryl sulphate.

Solid compositions may be obtained by conventional methods of blending, filling, tabletting or the like. Repeated blending operations may be used to distribute the active agent throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule, for example of gelatin containing the compound, if desired with a carrier or other excipients.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils, for example almond oil, fractionated coconut oil, oily esters, for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

The compounds of this invention may also be administered by a non-oral route. In accordance with routine pharmaceutical procedure, the compositions may be formulated, for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as a bottle from which the appropriate dose may be withdrawn or a solid form or concentrate which can be used to prepare an injectable formulation.

The compounds of this invention may also be administered by inhalation, via the nasal or oral routes. Such administration can be carried out with a spray formulation comprising a compound of the invention and a suitable carrier, optionally suspended in, for example, a hydrocarbon propellant.

Preferred spray formulations comprise micronised compound particles in combination with a surfactant, solvent or a dispersing agent to prevent the sedimentation of suspended particles. Preferably, the compound particle size is from about 2 to 10 microns.

A further mode of administration of the compounds of the invention comprises transdermal delivery utilising a skin-patch formulation. A preferred formulation comprises a compound of the invention dispersed in a pressure sensitive adhesive which adheres to the skin, thereby permitting the compound to diffuse from the adhesive through the skin for delivery to the patient. For a constant rate of percutaneous absorption, pressure sensitive adhesives known in the art such as natural rubber or silicone can be used.

As mentioned above, the effective dose of compound depends on the particular compound employed, the condition of the patient and on the frequency and route of administration. A unit dose will generally contain from 20 to 1000 mg and preferably will contain from 30 to 500 mg, in particular 50, 100, 150, 200, 250, 300, 350, 400, 450, or 500 mg. The composition may be administered once or more times a day for example 2, 3 or 4 times daily, and the total daily dose for a 70 kg adult will normally be in the range 100 to 3000 mg. Alternatively the unit dose will contain from 2 to 20 mg of active ingredient and be administered in multiples, if desired, to give the preceding daily dose.

No unacceptable toxicological effects are expected with compounds of the invention when administered in accordance with the invention.

The present invention also provides a method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) or a pharmaceutically acceptable salt or solvate thereof.

The activity of the compounds of the present invention, as NK₃ ligands, is determined by their ability to inhibit the binding of the radiolabelled NK₃ ligands, [¹²⁵I]-[Me-Phe⁷]-NKB or [³H]-Senktide, to guinea-pig and human NK₃ receptors (Renzetti et al, 1991, Neuropeptide, 18, 104-114; Buell et al, 1992, FEBS, 299(1), 90-95; Chung et al, 1994, Biochem. Biophys. Res. Commun., 198(3), 967-972).

The binding assays utilized allow the determination of the concentration of the individual compound required to reduce by 50% the [¹²⁵I]-[Me-Phe⁷]-NKB and [³H]-Senktide specific binding to NK₃ receptor in equilibrium conditions (IC₅₀).

Binding assays provide for each compound tested a mean IC₅₀ value of 2-5 separate experiments performed in duplicate or triplicate. The most potent compounds of the present invention show IC₅₀ values in the range 0.1-1000 nM. The NK₃-antagonist activity of the compounds of the present invention is determined by their ability to inhibit senktide-induced contraction of the guinea-pig ileum (Maggi et al, 1990, Br. J. Pharmacol, 101, 996-1000) and rabbit isolated iris sphincter muscle (Hall et al., 1991, Eur. J. Pharmacol., 199, 9-14) and human NK₃ receptors-mediated Ca⁺⁺ mobilization (Mochizuki et al, 1994, J. Biol. Chem., 269, 9651-9658). Guinea-pig and rabbit in-vitro functional assays provide for each compound tested a mean K_{B} value of 3-8 separate experiments, where K_{B} is the concentration of the individual compound required to produce a 2-fold rightward shift in the concentration-response curve of senktide. Human receptor functional assay allows the determination of the concentration of the individual compound required to reduce by 50% (IC₅₀ values) the Ca⁺⁺ mobilization induced by the agonist NKB. In this assay, the compounds of the present invention behave as antagonists.

The activity of the compounds of the present invention, as NK-2 ligands, is determined by their ability to inhibit the binding of the radiolabelled NK-2 ligands, [¹²⁵I]-NKA or [³H]-NKA, to human NK-2 receptors (Aharony et al, 1992, Neuropeptide, 23, 121-130).

The binding assays utilized allow the determination of the concentration of the individual compound required to reduce by 50% the [¹²⁵I]-NKA and [³H]-NKA specific binding to NK2 receptor in equilibrium conditions (IC₅₀).

Binding assays provide for each compound tested a mean IC₅₀ value of 2-5 separate experiments performed in duplicate or triplicate. The most potent compounds of the present invention show IC₅₀ values in the range 0.5-1000 nM, such as 1-1000 nM. The NK-2-antagonist activity of the compounds of the present invention is determined by their ability to inhibit human NK-2 receptor-mediated Ca⁺⁺ mobilization (Mochizuki et al, 1994, J. Biol. Chem., 269, 9651-9658). Human receptor functional assay allows the determination of the concentration of the individual compound required to reduce by 50% (IC₅₀ values) the Ca⁺⁺ mobilization induced by the agonist NKA. In this assay, the compounds of the present invention behave as antagonists.

The therapeutic potential of the compounds of the present invention in treating the conditions can be assessed using rodent disease models.

As stated above, the compounds of formula (I) are also considered to be useful as diagnostic tools. Accordingly, the invention includes a compound of formula (I) for use as diagnostic tools for assessing the degree to which neurokinin-3 and neurokinin-2 receptor activity (normal, overactivity or underactivity) is implicated in a patient's symptoms. Such use comprises the use of a compound of formula (I) as an antagonist of said activity, for example including but not restricted to tachykinin agonist-induced inositol phosphate turnover or electrophysiological activation, of a cell sample obtained from a patient. Comparison of such activity in the presence or absence of a compound of formula (I), will disclose the degree of NK-3 and NK-2 receptor involvement in the mediation of agonist effects in that tissue.

The following Descriptions illustrate the preparation of the intermediates, whereas the following Examples illustrate the preparation of the compounds of the invention.

### DESCRIPTIONS AND EXAMPLES

Description A: 3-Methyl-2-phenyl-quinoline-4-carbonyl Chloride

A solution of 14.35 g (54.5 mmol) of 3-methyl-2-phenyl-quinoline-4-carboxylic acid (CAS [43071-45-0]) and one drop of DMF in 100 ml methylene chloride was treated dropwise with 6.92 g (54.5 mmol) oxalyl chloride. After the end of the gas evolution the mixture was concentrated to dryness and used in the next step without further purification.

C₁₇H₁₂ClNO

MW 281.79

Description B: 3-Methyl-2-phenyl-quinoline-4-carboxylic Acid Methyl Ester

32.12 g (114 mmol) of crude 3-methyl-2-phenyl-quinoline-4-carbonyl chloride (compound of Description A) were suspended in 100 ml of CH₂Cl₂ and 100 ml of MeOH, dissolved in 400 ml of CH₂Cl₂, were added dropwise. After stirring for 18 h, the solvent was evaporated in vacuo to dryness, the residue was taken up with CH₂Cl₂ and washed with 1% NaHCO₃; the organic layer was dried over Na₂SO₄, filtered and evaporated in vacuo to dryness to yield 31.6 g of the title compound as a solid, which was used in the following reaction without further purification.

C₁₈H₁₅NO₂

MW 277.31

MP=73-75° C.

IR (KBr) 3441, 3051, 2954, 1731, 1582, 1556 cm⁻¹.

Description C: 3-Methyl-2-phenyl-quinoline-4-carboxylic Acid Tert-butyl Ester

15.31 g (54.5 mmol) of crude 3-methyl-2-phenyl-quinoline-4-carbonyl chloride (compound of Description A) were dissolved in 100 ml anhydrous THF. This mixture was added dropwise to a solution of 6.12 g (5.45 mmol) potassium terbutylate in 100 ml anhydrous THF and stirred for 16 h. The reaction mixture was neutralised with acetic acid and the solvent concentrated. The residue was dissolved in AcOEt and the organic phase was washed with water and dried over MgSO₄. After concentration to dryness the residue was dissolved in heptane and filtered. The mother liquors were then purified by flash chromatography over silicagel (eluent: heptane/CH₂Cl₂: ½) affording 3 g (17.2%) of the desired ester.

C₂₁H₂₁NO₂

MW=319.40

¹H NMR (CDCl₃) δ: 1.72 (s, 9H); 2.42 (s, 3H); 7.40-7.88 (m, 8H ar); 8.15 (d, 1H ar)

Description D: 3-Bromomethyl-2-phenyl-quinoline-4-carboxylic Acid Methyl Ester

10 g (36 mmol) of 3-methyl-2-phenyl-quinoline-4-carboxylic acid methyl ester (compound of Description B) were dissolved in 500 ml of CH₃CN; 13 g (72 mmol) of N-bromosuccinimide were added and the reaction mixture was heated to reflux. After adding 1 g (4.1 mmol) of dibenzoylperoxide, the reaction was refluxed for 24 h; then additional 4 g (22.5 mmol) of N-bromosuccinimide and 0.5 g (2.0 mmol) of dibenzoylperoxide were added and the reaction was refluxed for 4 h. The solvent was evaporated in vacuo to dryness to yield 26.1 g of crude methyl 3-bromomethyl-2-phenylquinoline-4-carboxylate (theorical amount, 12.8 g) which was used in the following reaction without further purification.

C₁₈H₁₄BrNO₂

MW=356.23

Description E: 3-Bromomethyl-2-phenyl-quinoline-4-carboxylic Acid Tert-butyl Ester

A solution of 3 g (9.4 mmol) of 3-methyl-2-phenyl-quinoline-4-carboxylic acid tert.-butyl ester (compound of Description C) and 0.3 g benzoyl peroxide in 100 ml acetonitrile was heated to reflux and 3.34 g (18.8 mmol) NBS were then added portionwise. The reflux was maintained one night, then the solvent was concentrated and the residue was triturated with 50 ml carbon tetrachloride and filtered. The filtrate was diluted with 50 ml methylene chloride and the organic phase was washed with water, a solution of NaHCO₃, again with water, dried over MgSO₄ and concentrated. The residue was purified by flash chromatography on silicagel (eluent: methylene chloride/heptane: 3/1) to afford 3 g (80%) of the title bromide as an oil.

C₂₁H₂₀BrNO₂

MW=398.30

¹H NMR (CDCl₃) δ: 1.77 (s, 9H); 4.67 (s, 2H); 7.40-7.85 (7H ar); 7.89 (d, 1H ar); 8.14 (d, 1H ar)

Description 1:3-[1,4′]Bipiperidinyl-1′-ylmethyl-2-phenyl-quinoline-4-Carboxylic Acid Methyl Ester

5 g (14 mmol) of 3-bromomethyl-2-phenyl-quinoline-4-carboxylic acid methyl ester (compound of Description B), 2.9 g, (15.4 mmol) of 90% 4-piperidino-piperidine (Aldrich), 2.7 ml (15.4 mmol) diisopropylethyl amine were dissolved in 100 ml of dry THF and the mixture was stirred for one night at 50° C. The solvent was concentrated, the residue was dissolved in methylene chloride, washed with water, and the organic phase was dried over MgSO₄. After concentration of the solvent the residue was purified by flash chromatography over 160 g of silicagel (eluent CH₂Cl₂/MeOH/NH₄OH: 95/5/0.5) affording 3.5 g (yield 56%) of the title compound as a white solid.

¹H NMR (CDCl₃) δ: 1.29-2.02 (12H); 2.25 (1H); 2.47 (4H); 2.78 (2H); 3.66 (2H); 4.05 (3H); 7.38-7.55 (5H ar); 7.58 (1H ar); 7.72 (1H ar); 7.88 (1H ar); 8.17 (1H ar)

Description 2: 3-[1,4′]-Bipiperidinyl-1′-ylmethyl-2-phenyl-quinoline-4-carboxylic Acid Dihydrochloride

3.5 g (7.9 mmol) of 3-[1,4′]bipiperidinyl-1′-ylmethyl-2-phenyl-quinoline-4-carboxylic acid methyl ester (compound of Description 1) and 50 ml 6N HCl are refluxed for 1.5 h then concentrated to dryness. The residue is triturated in acetone. This process is re-applied twice to the solid thus obtained affording, after drying in vacuo 4.5 g of the title compound as a crude dihydrochloride used without further purification in the next step.

¹H NMR (DMSO-d₆)δ: 1.16-2.29 (10H); 2.62-3.38 (8H); 4.46 (2H); 5.77 (1H exch with D₂O); 7.45-8.30 (9H ar); 11.12 (1H exch with D₂O)

Description 3: 4-(1-Benzyl-piperidin-4-yl)-piperazine-1-carboxylic acid 9H-fluoren-9-ylmethyl Ester

The pH of a solution of 3.38 g (9.8 mmol) of fmoc-piperazine hydrochloride (RN 215190-22-0) and 2.042 g (10.8 mmol) of 1-benzyl-4-piperidone in 40 ml of methanol was adjusted at approximately 5.7 by mean of acetic acid. Then 493 mg (7.8 mmol) of cyanoborohydride was added potionwise while maintaining the pH between 5 and 6. The reaction was controlled by TLC and 0.5 g benzyl piperidone was added twice, after each time 2 h stirring. The mixture was left overnight, then concentrated. The residue was treated at 0° C. by 20 ml aqueous NaOH 0.5N and extracted with 50 ml of AcOEt. The organic phase was washed twice with 50 ml of water, dried over MgSO₄ and concentrated.

The residue was purified by flash chromatography (silica gel, first CH₂Cl₂ then CH₂Cl₂/MeOH: 98/2 and 95/5 to finish) to afford 3.35 g (yield 71%) of the title compound.

¹H NMR (DMSO-d₆) δ: 7.76 (2H, dd), 7.57 (2H, dd), 7.48-7.20 (9H, m), 4.43 (2H, d), 4.23 (1H, t), 3.58 (2H, s), 3.49 (4H, m), 3.02 (2H, m), 2.50 (4H, m), 2.32 (1H, m), 2.05 (2H, m), 1.89-1.52 (411, m)

Description 4: 4-Piperidin-4-yl-piperazine-1-carboxylic acid 9H-fluoren-9-ylmethyl Ester

Chloroethylchloroformate (192 mg, 1.3 mmol) was added to an ice cooled solution of 500 mg (1 mmol) of 4-(1-benzyl-piperidin-4-yl)-piperazine-1-carboxylic acid 9H-fluoren-9-ylmethyl ester (compound of Description 3) in 15 ml methylene chloride. The mixture was stirred at room temperature for 2 h and left overnight in the deep freezer. The mixture was concentrated to dryness, 10 ml of methanol were added and the white suspension was refluxed for 1 h After concentration the residue was triturated with ether. The white solid was filtered, washed with ether and dried affording 350 mg (yield 81%) of hydrochloride of the title compound.

¹H NMR (DMSOd₆) δ: 11.65 (1H, br), 9.18 (111, br), 8.93 (1H, br), 7.91 (2H, d), 7.64 (2H, d), 7.44 (2H, t), 7.35 (2H, t), 4.41 (2H, d), 4.30 (1H, t), 3.99 (1H, m), 3.64-3.19 (8H, m), 2.92 (4H, m), 2.29 (2H, m), 1.98 (2H, m)

Description 5: 3-{4-[4-(9H-Fluoren-9-ylmethoxycarbonyl)-piperazin-1-yl]-piperidin-1-ylmethyl}-2-phenyl-quinoline-4-carboxylic Acid Methyl Ester

A suspension/solution of 0.35 g (0.9 mmol) of crude 4-piperidin-4-yl-piperazine-1-carboxylic acid 9H-fluoren-9-ylmethyl ester (compound of Description 4), 0.32 g (0.9 mmol) of methyl 3-bromomethyl-2-phenylquinoline-4-carboxylate and 0.58 g (4.5 mmol) of DIEA (diethylisopropylamine) in 5 ml THF was stirred 18 h at room temperature. After concentration of the solvent the residue was dissolved in 10 ml of AcOEt plus 10 ml of water. The organic phase was washed with water, dried over MgSO₄ and concentrated.

The residue was purified by flash chromatography (silica gel, CH₂Cl₂/MeOH: 96/4) to afford 0.26 g (yield 42.5%) of the title compound.

¹H NMR (CDCl₃) δ: 8.16 (1H, d), 7.90 (1H,dd), 7.81-7.67 (3H, m), 7.60-7.28 (12H, m), 4.41 (2H, d), 4.23 (1H, t), 4.06 (3H, s), 3.68 (2H, s), 3.46 (4H, m), 2.77 (2H, m), 2.45 (4H, m), 2.15 (1H, m), 1.89 (2H, m), 1.66 (2H, m), 1.46 (2H, m)

Description 6: 3-{4-[4-(9H-Fluoren-9-ylmethoxycarbonyl)-piperazin-1-yl]-piperidin-1-ylmethyl}-2-phenyl-quinoline-4-carboxylic Acid

A solution of 214 mg (0.32 mmol) of 3-{4-[4-(9H-fluoren-9-ylmethoxycarbonyl) -piperazin-1-yl]-piperidin-1-ylmethyl}-2-phenyl-quinoline-4-carboxylic acid methyl ester (compound of Description 5) in 10 ml 6N aqueous hydrochloric acid was refluxed for 2 h. The solution was concentrated, the residue was suspended in acetone and the solvent concentrated again to afford the crude title compound as hydrochloride which was used in the next step without further purification.

¹H NMR (DMSO-d₆) δ: 11.70 (1H, br), 8.17 (1H, d), 8.07-7.50 (11H, m), 7.49-7.22 (4H, m), 4.51 (2H, br), 4.37 (2H, d), 4.27 (1H, t), 3.90 (2H, m), 3.59-2.55 (13H, m), 2.09 (2H, m), 1.64 (2H, m)

Description 7: 4-{1-[2-Phenyl-4-((S)-1-phenyl-propylcarbamoyl)-quinolin-3-ylmethyl]-piperidin-4-yl}-piperazine-1-carboxylic Acid 9H-fluoren-9-ylmethyl Ester

A mixture of the crude 4-{1-[2-phenyl-4-((S)-1-phenyl-propylcarbamoyl)-quinolin-3-ylmethyl]-piperidin-4-yl}-piperazine-1-carboxylic acid 9H-fluoren-9-ylmethyl ester of Description 6 (0.32 mmol), 182 mg (0.48 mmol) of HBTU, 162 mg (1.6 mmol) of triethylamine, 65 mg (0.48 mmol) of (S)-(−)-1-phenylpropylamine, 5 ml of THF and 3 ml of CH₂Cl₂ stabilised over amylene, was stirred at room temperature for 20 h. The solvent was concentrated and the residue dissolved in 10 ml of water and 10 ml of AcOEt. The organic phase was washed with 0.5 N aqueous NaOH the 4 times with 10 ml of water and dried over MgSO₄. After concentration of the solvent the residue was purified by flash chromatography (silica gel, CH₂Cl₂/MeOH: 98/2) to afford 68 mg (yield 27.5%) of the title compound.

¹H NMR (CDCl₃) δ: 8.70 (1H, d br), 8.18-8.00 (2H, m), 7.75 (2H, d), 7.56 (2H, d), 7.51-7.18 (16H, m), 5.32 (1H, m), 4.41 (2H, d), 4.23 (1H, t), 3.59 (2H,s), 3.42 (4H, m), 2.54 (1H, m), 2.31 (4H, m), 2.22-1.86 (4H, m), 1.70-1.21 (6H, m), 1.05 (3H, t)

Description 8: 3-[4-(9H-Fluoren-9-ylmethoxycarbonylamino)-piperidin-1-ylmethyl]-2-phenyl-quinoline-4-carboxylic Acid Methyl Ester

A mixture of 1.87 g (5.2 mmol) of 3-bromomethyl-2-phenyl-quinoline-4-carboxylic acid methyl ester (compound of Description D), 2.07 (5.8 mmol) of (fmoc-4-amino)-piperidine hydrochloride, 1.49 g (11.5 mmol) of diisopropylethylamine, 1 g of potassium fluoride and 45 ml of THF was stirred at reflux for 4 h. The reaction mixture was concentrated to dryness and dissolved in 40 ml of AcOEt and 40 ml of water. The aqueous phase was extracted three times with CH₂Cl₂ and the organic phases were pooled. The organic phase was dried over MgSO₄ and concentrated to dryness. The residue was purified by flash chromatography on silicagel (eluent, heptane/AcOEt: 4/1) to afford 2.3 g of the title compound (yield 73.9%).

¹H NMR: (CDCl₃) δ: 1.12-1.47 (m, 2H); 1.83 (m, 2H); 2.05 (m, 2H); 2.63 (m, 2H); 3.45 (m, 1H); 3.69 (s, 2H); 4.04 (s, 3H); 4.17 (t, 1H); 4.37 (d, 2H); 4.62 (d br, 1H); 7.22-7.82 (m, 15H ar); 7.90 (dd, 1H ar); 8.16 (dd, 1H ar)

Description 9: 3-[4-(9H-Fluoren-9-ylmethoxycarbonylamino)-piperidin-1-ylmethyl]-2-phenyl-quinoline-4-carboxylic Acid

A solution of 2.2 g (3.8 mmol) of 3-[4-(9H-fluoren-9-ylmethoxycarbonylamino)-piperidin-1-ylmethyl]-2-phenyl-quinoline-4-carboxylic acid methyl ester (compound of Description 8) in 30 ml of 6 N aqueous hydrochloric acid was refluxed for 2 h. The solution was concentrated to dryness in vacuo. Acetone was added to the residue and evaporated to remove azeotropically the water. The process was repeated three times and the final residue was dried in vacuo at 50° C., affording 2.34 g of crude acid used without further purification in the next step.

¹H NMR: (DMSO-d₆)δ: 1.32-1.83 (m, 4H); 2.72-3.18 (m, 4H); 3.45 (m, 1H); 4.02-4.45 (m br, 5H); 4.50 (s, 2H); 7.23-7.48 (m, 4H ar); 7.52-8.07 (m, 11H ar); 8.13-8.17 (2H ar)

Description 10: {1-[4-((S)-1-Cyclohexyl-ethylcarbamoyl)-2-phenyl-quinolin-3-ylmethyl]-piperidin-4-yl}-carbamic Acid 9H-fluoren-9-ylmethyl Ester

A mixture of 400 mg (0.7 mmol) of 3-[4-(9H-fluoren-9-ylmethoxycarbonylamino)-piperidin-1-ylmethyl]-2-phenyl-quinoline-4-carboxylic acid (compound of Description 9), 398 mg (1.05 mmol) of BTU, 283 mg (2.8 mmol) of triethylamine, 133 mg (1.05 mmol) of (S)-1-cyclohexyl-ethylamine, 10 ml of anhydrous THF and 6 ml of CH₂Cl₂ stabilised with amylene was stirred for 24 h at room temperature. The mixture was concentrated and the residue was dissolved in 10 ml of AcOEt and 10 ml of water. The organic phase was washed with 10 ml of 0.5 N aqueous NaOH then with water until neutral. The organic phase was dried over MgSO₄ and concentrated to dryness. The residue was purified by flash chromatography on silicagel (eluent, CH₂Cl₂/MeOH: 99/1) to afford 147 mg (30%) of the title compound which was used without further purification in the next step.

¹H NMR (DMSO-d₆) δ: 0.90-1.92 (m, 151H); 1.17 (d, 3H); 2.42 (m, 2H); 3.09 (m, 1H); 3.43 (m, 2H); 3.52 (s, 2H); 4.02 (m, 1H); 4.21 (m, 3H); 4.36 (m, 1H); 7.14-7.95 (m, 16H ar); 8.04 (1H ar); 8.56 (br, 1H)

Description 11: 3-(2-Oxo-[1,4′]-bipiperidinyl-1′-ylmethyl)-2-phenyl-quinolin-4-carboxylic Acid Methyl Ester

A mixture of 0.25 g (1.4 mmol) of [1,4′-bipiperidine]-2-one (RN 159874-26-7), 0.5 g (1.4 mmol) of 3-bromomethyl-2-phenyl-quinoline-4-carboxylic acid methyl ester (compound of description D), 0.5 g of potassium fluoride, 0.76 g (4.2 mmol) of DIEA and 15 ml of THF was stirred at room temperature for 18 h. The solvent was concentrated and the residue dissolved in methylene chloride. The organic phase was washed with water and dried over MgSO₄. After concentration, the residue was purified by flash chromatography (silica gel CH₂Cl₂/MeOH/NH₄OH: 95/5/0.5) to afford 0.5 g (yield 77%) of the title compound.

¹H NMR (CDCl₃) δ: 8.15 (1H, dd), 7.89 (1H, dd), 7.79 (1H, td), 7.57 (1H, td), 7.48 (5H, m), 4.41 (1H, m), 4.06 (3H, s), 3.69 (2H, s), 3.16 (2H, m), 2.76 (2H, m), 2.34 (2H, m), 2.05 (2H, m), 1.87-1.38 (8H, m)

Description 12: 3-[4-(2-Oxo-pyrrolidin-1-yl)-piperidin-1-ylmethyl]-2-phenyl -quinoline-4-carboxylic Acid Methyl Ester

This compound was prepared using 1-(4-piperidinyl)-2-piperidone following the procedure of Description 11. The title compound was obtained in 83% yield.

¹H NMR (CDCl₃) δ: 8.15 (1H, dd), 7.87 (1H, dd), 7.73 (1H, td), 7.55-7.38 (6H, m), 4.06 (3H, s), 3.87 (1H, m), 3.69 (2H, s), 3.33 (2H, t), 2.75 (2H, m), 2.37 (2H, t), 2.13-1.88 (4H,m), 1.77-1.42 (4H, m)

Description 13: 3-(2-Oxo-[1,4′]bipiperidinyl-1′-ylmethyl)-2-phenyl-quinoline-4-carboxylic Acid

A solution of 0.5 g of crude 3-(2-oxo-[1,4′]bipiperidinyl-1′-ylmethyl)-2-phenyl -quinoline-4-carboxylic acid methyl ester (compound of Description 11) in 10 ml of 6N hydrochloric acid was refluxed for 2 h. The acid was concentrated and the residue was washed three time with a small amount of acetone to afford, after drying, 0.55 g of crude title compound hydrochloride which was used without further purification in the next step.

Description 14: 3-[4-(2-Oxo-pyrrolidin-1-yl)-piperidin-1-ylmethyl]-2-phenyl -quinoline-4-carboxylic Acid

Applying the procedure of Description 3 to 0.55 g of 3-[4-(2-oxo-pyrrolidin-1-yl) -piperidin-1-ylmethyl]-2-phenyl-quinoline-4-carboxylic acid methyl ester (compound of Description 12) afforded 0.64 g (approx. 100%) of crude title compound as hydrochloride which was used without further purification in the next step.

Description 15: [1,4′]Bipiperidinyl-3,1′-dicarboxylic acid 1′-tert-butyl ester 3-ethyl ester

Procedure related to J. Org. Chem. 1990, 55, 2552-4.

A mixture of 2 g (10 mmol) 1-ter-butoxycarbonyl-4-piperidone, 1.6 g (10 mmol) ethylnipecotate and 3.72 ml (12.5 mmol) titanium IV isopropoxyde was stirred at room temperature for 3 h. Ethanol (10 ml) was added followed by 0.42 g (6.7 mmol) of sodium cyano borohydride and stirring was continued for 16 h.

The mixture was treated with 2 ml of water and the solid was filtered off using a filtration aid (i.e. Clarcel®). The filtration cake was washed twice with ethanol and all solvent fractions were mixed together and concentrated. The residue was taken-up with AcOEt. The insoluble fraction was filtered off on Clarcel® and the solution concentrated in vacuo. The residue was purified by flash chromatography on silicagel (200 g) (eluent: AcOEt/MeOH: 95/5) to afford 0.92 g (28%) of the title compound.

¹H-NMR (DMSO-d₆)δ: 1.17 (t, 3H); 1.38 (s, 9H); 1.25-1.90 (m, 8H); 2.14-3.00 (m, 9H); 3.98 (m, 1H); 4.05 (q, 2H)

Description 16: [1,4′]Bipiperidinyl-3-carboxylic Acid Ethyl Ester, Bis Trifluoroacetate.

5 ml trifluoroacetic acid were added dropwise to a solution of 0.9 g (2.75 mmol) of [1,4′]bipiperidinyl-3,1′-dicarboxylic acid 1′-tert-butyl ester 3-ethyl ester (compound of Description 15) in 5 ml of methylene chloride and the mixture was stirred for 1 h at room temperature. After concentration of the solvent the residue was washed repeatedly with diethyl ether affording 1.25 g (93.8%) of the title compound as a thick oil.

¹H-NMR (DMSO-d₆): 1.20 (t, 3H); 1.40-2.10 (m, 6H); 2.19 (m, 2H); 2.68-3.60 (m, 10H); 4.12 (q, 2H); 8.65 (broad band, 1H); 8.94 (broad band, 1H); 10.2 (broad band, 1H)

Description 17: 1′-(4-tert-Butoxycarbonyl-2-phenyl-quinolin-3-ylmethyl)-[1,4]bipiperidinyl-3-carboxylic Acid Ethyl Ester.

A solution of 0.2 g (0.5 mmol) of crude 3-bromomethyl-2-phenyl-quinoline-4-carboxylic acid tert.-butyl ester (compound of Description D) (a batch at 70%, corresponding to 0.35 mmol), 0.19 g (0.53 mmol) [1,4′]bipiperidinyl-3-carboxylic acid ethyl ester, (compound of Description 16), 175 microliters (129 mg, 1 mmol) DIEA in THF (5 ml) was stirred at room temperature for 16 h. A TLC showed that the reaction was not completed, therefore 100 mg KF were added and the mixture was stirred at 50° C. for additional 4 h. The solvent was concentrated, the residue dissolved in AcOEt, the organic phase was washed with water, dried over MgSO₄ and concentrated again. The residue was purified by flash chromatography on silicagel (30 g) (eluent: first CH₂Cl₂ then CH₂Cl₂/MeOH: 95/5) affording 0.105 g (53.8%) of the title compound.

¹H-NMR (CDCl₃) δ: 1.20 (t, 3H); 1.31-3.08 (m, 1811); 1.71 (s, 9H); 3.61 (s, 2H); 4.11 (q, 2H); 7.38-7.55 (m, 5H ar); 7.58 (td, 1H ar); 7.70 (td, 1H ar); 7.90 (dd, 1H ar); 8.14 (dd, 1H ar)

Description 18: 1′-(4-Carboxy-2-phenyl-quinolin-3-ylmethyl)-[1,4′]bipiperidinyl-3-carboxylic Acid Ethyl Ester

A mixture of 100 mg (0.13 mmol) of 1′-(4-tert-butoxycarbonyl-2-phenyl-quinolin-3-ylmethyl)-[1,4′]bipiperidinyl-3-carboxylic acid ethyl ester (compound of Description 17), 1 ml methylene chloride and 0.5 ml trifluoroacetic acid (TFA) was stirred at room temperature for 2 h. The solvent was concentrated and the residue was triturated with diethyl ether, filtered, triturated again, filtered and dried in vacuum affording 0.106 g of the title compound as ditrifluoroacetate.

¹H-NMR (CDCl₃) δ: 1.23 (t, 3H); 1.49 (m, 1H); 1.87-2.31 (m, 6H); 2.45 (m, 2H); 2.70 (m, 2H); 2.95-3.68 (6H); 4.13 (q, 2H); 4.25 (s, 1H); 7.10 (broad band, 1H), 7.40-7.51 (5H ar); 7.66 (td, 1H ar); 7.83 (td, 1H ar); 8.19 (dd, 1H ar); 8.26 (dd, 1H ar)

Description 19: 7-Methoxy-3-methyl-2-phenyl-quinoline-4-carboxylic Acid Methyl Ester

16 g (54.5 mmol) of 7-methoxy-3-methyl-2-phenyl-quinoline-4-carboxylic acid (prepared analogously to starting material of Description A) were suspended in 400 ml of dry CH₂Cl₂ and 9.52 ml (126.93 mmol) of oxalyl chloride were added dropwise. Two drops of N,N-dimethylformamide (DMF) were added and the reaction mixture was stirred for 3 h at room temperature. The solvent was evaporated in vacuo to dryness, the residue was taken up with 150 ml of CH₂Cl₂ and quickly dropped in a solution of 200 ml of MeOH and 200 ml of CH₂Cl₂. After stirring for 1 h, the solvent was evaporated in vacuo to dryness, the residue was taken up with EtOAc and washed with 1% NaHCO₃; the organic layer was dried over Na₂SO₄, filtered and evaporated in vacuo to dryness. After trituration of the residue with Et₂O, 19 g of the title compound were recovered as a dark powder used without further purification.

IR (KBr) 3067, 2947, 1918, 1729, 1634, 1581, 1246, 846 cm⁻¹

Description 20: 3-[1,4′]Bipiperidinyl-1′-ylmethyl-8-bromo-7-methoxy-2-phenyl -quinoline-4-carboxylic Acid Methyl Ester

Prepared as described in Description B and Description 1 from 4.7 g (15.3 mmol) of 7-methoxy-3-methyl-2-phenyl-quinoline-4-carboxylic acid methyl ester (compound of Description 19), 5.5 g (30.6 mmol) of N-bromosuccinimide, 0.5 g (2.05 mmol) of dibenzoylperoxide, 3.85 g (23 mmol) of 4-piperidinopiperidine and 3.18 g (23.0 mmol) of K₂CO₃, by stirring in CH₃CN at room temperature for 4 h. The title compound (6.2 g) was obtained.

IR (KBr) 3370, 2938, 1712, 1612, 1352, 1268, 1174, 704 cm⁻¹.

Description 21: 3-[1,4′]Bipiperidinyl-1′-ylmethyl-8-bromo-7-methoxy-2-phenyl -quinoline-4-carboxylic Acid Hydrochloride

Prepared as described in Description 2 from 6.0 g (10.9 mmol) of 3-[1,4′]bipiperidinyl-1′-ylmethyl-8-bromo-7-methoxy-2-phenyl-quinoline-4-carboxylic acid methyl ester (compound of Description 20) and 50 ml of 6 N HCl yielding 4.7 g of a slightly brown powder.

IR: (KBr) 3453, 2939, 2532, 1714, 1607, 1598, 1271, 1072, 960, 779, 705, cm⁻¹.

Description 22: 3-[1,4′]Bipiperidinyl-1′-ylmethyl-8-bromo-7-hydroxy-2-phenyl -quinoline-4-carboxylic Acid Hydrobromide

5.5 g (9.95 mmol) of 3-[1,4′]Bipiperidinyl-1′-ylmethyl-8-bromo-7-methoxy-2-phenyl -quinoline-4-carboxylic acid methyl ester (compound of Description 20) were dissolved in 100 ml of 48% HBr and the solution was refluxed for 6 h. The solvent was evaporated in vacuo to dryness, yielding 7.2 g of a dark powder which was used in following reactions without further purification.

C₂₇H₃₀BrN₃O₃.2HBr

MW=686.28

Description 23: 3-[1,4′]Bipiperidinyl-1′-ylmethyl-7-hydroxy-2-phenyl -quinoline-4-carboxylic Acid ((S)-1-phenyl-propyl)-amide

1.7 g (2.48 mmol) of 3-[1,4′]Bipiperidinyl-1′-ylmethyl-8-bromo-7-hydroxy-2-phenyl -quinoline-4-carboxylic acid hydrobromide (compound of Description 22), 0.67 g (4.96 mmol) of(S)-1-phenylpropylamine, 1.88 g (4.96 mmol) of HBTU and 1.38 ml (9.92 mmol) of TEA were dissolved in a 1:1 mixture of CH₂Cl₂ and THF and the reaction mixture was stirred at 50° C. for 4 hours then allowed to cool to room temperature and stirred overnight. The solvent was evaporated in vacuo to dryness and the residue dissolved in AcOEt. The organic phase was washed three times with NH₄OH, then water, dried over Na₂SO₄ and evaporated in vacuo to dryness. The residue, dissolved in EtOH (100 ml) in presence of 10% Pd/C (20 mg) and TEA (6 ml), was hydrogenated at 5 psi for 2 h. The suspension was filtered and evaporated to dryness and then purified by flash chromatography over silicagel (eluent EtOAc/MeOH/NH₄OH: 90/10/1). The crude compound was triturated in Et2O affording 0.23 g of the title compound as a yellow powder.

[a]_{D}²⁰=−41.88 (c=0.22, MeOH)

Description 24: 7-Chloro-3-methyl-2-phenyl-quinoline-4-carboxylic Acid

6-Chloroisatin (3.3 g, 18 mmol), [CAS 6341-92-0], was dissolved in EtOH (100 ml) containing KOH (4.7 g). After stirring the solution 30 min at room temperature, propiophenone (2.4 g, 18 mmol) was added and the solution was refluxed for 4 h the solvent was evaporated to dryness and the residue was dissolved in water (200 ml), washed with Et₂O and then acidified with citric acid. The precipitated obtained was filtered and dried to give 5 g of the title compound as beige powder that was used in the next step without further purification.

C₁₇H₁₂ClNO₂

MW=297.74

Description 25: 7-Chloro-3-methyl-2-phenyl-quinoline-4-carboxylic Acid ((S)-1-cyclohexyl-ethyl)-amide

7-Chloro-3-methyl-2-phenyl-quinoline-4-carboxylic acid (2.9 g, 9.7 mmol) (prepared as described in Description 24) was suspended in CH₂Cl₂ (60 ml) and oxalyl chloride (2.5 ml, 28.6 mmol) was added dropwise at 0° C. under magnetic stirring. After 15 min 2 drops of DMF were added. The reaction was vigorous with gas evolution. The mixture was stirred at room temperature until the solid was completely dissolved (about 3 h). The solution was evaporated. The crude material was re-dissolved in CH₂Cl₂ (20 ml) and slowly dropped into a suspension of K₂CO₃ (4 g) and (S)-1-cyclohexylethyl amine (2.5 ml, 16.8 mmol) in TIF (60 ml) maintaining the temperature between 10-15° C. The dark solution was left 1 h at room temperature and 1 h refluxing. The organic phase was then washed with water, NaHCO₃, brine, dried over Na₂SO₄ and then evaporated under vacuum. The crude residue was triturated with iPr₂O. After filtration 1.6 g of the title compound were obtained, mp=204-207° C. Yield: 41%

Description 26: 3-Bromomethyl-7-chloro-2-phenyl-quinoline-4-carboxylic Acid ((S)-1-cyclohexyl-ethyl)-amide

7-Chloro-3-methyl-2-phenyl-quinoline-4-carboxylic acid ((S)-1-cyclohexyl-ethyl)-amide (1.5 g, 4.8 mmol; compound prepared as in Description 25) and N-bromosuccinimmide (1.5 g, 8.4 mmol) were suspended in CCl₄ (30 ml) and warmed to incipient reflux. Dibenzoyl peroxide (about 30 mg) was carefully added portionwise and the solution was then refluxed for 2 h. The solvent was removed under vacuum and the residue was re-dissolved in CH₂Cl₂ (200 ml) and filtered. DCM was then evaporated and the residue was triturated in Et₂O to give 0.4 g of the title compound as a powder that were in the next step used without further purification.

Description 27: 3-Bromomethyl-7-fluoro-2-phenyl-quinoline-4-carboxylic Acid ((S)-1-cyclohexyl-ethyl)-amide

3-Bromomethyl-7-fluoro-2-phenyl-quinoline-4-carboxylic acid ((S)-1-cyclohexyl-ethyl)-amide was prepared starting from 6-fluoroisatine [CAS 324-03-8] and propiophenone following the procedures described in Description 24-26.

Description 28: 3-Bromomethyl-8-fluoro-2-phenyl-quinoline-4-carboxylic Acid ((S)-1-cyclohexyl-ethyl)-amide

3-Bromomethyl-8-fluoro-2-phenyl-quinoline-4-carboxylic acid ((S)-1-cyclohexyl-ethyl) -amide was prepared starting from 7-fluoroisatine [CAS 317-204] and propiophenone following the procedures described in Description 24-26.

Description 29: 3-Bromomethyl-6-fluoro-2-phenyl-quinoline-4-carboxylic Acid ((S)-1-cyclohexyl-ethyl)-amide

3-Bromomethyl-6-fluoro-2-phenyl-quinoline-4-carboxylic acid ((S)-1-cyclohexyl-ethyl) -amide was prepared starting from 5-fluoroisatine and propiophenone following the procedures described in Description 24-26.

Description 30: 3-Bromomethyl-2-thiophen-2-yl-quinoline-4-carboxylic Acid ((S)-1-cyclohexyl-ethyl)-amide

3-Bromomethyl-2-thiophen-2-yl-quinoline-4-carboxylic acid ((S)-1-cyclohexyl-ethyl) -amide was prepared starting from isatine and 1-(2-thienyl)-1-propanone following the procedures described in Description 24-26.

Description 31: 3-Bromomethyl-2-(2-fluoro-phenyl)-quinoline-4-carboxylic Acid ((S)-1-cyclohexyl-ethyl)-amide

3-Bromomethyl-2-(2-fluoro-phenyl)-quinoline-4-carboxylic acid ((S)-1-cyclohexyl-ethyl) -amide was prepared starting from isatine and (2-fluoro)propiophenone following the procedures described in Description 24-26.

Description 32: 3-Bromomethyl-2-(4-fluoro-phenyl)-quinoline-4-carboxylic Acid ((S)-1-cyclohexyl-ethyl)-amide

3-Bromomethyl-2-(4-fluoro-phenyl)-quinoline-4-carboxylic acid ((S)-1-cyclohexyl-ethyl)-amide was prepared starting from isatine and (4-fluoro)propiophenone following the procedures described in Description 24-26.

Description 33: 3-Bromomethyl-2-(4-trifluoromethyl-phenyl)-quinoline-4-carboxylic acid ((S)-1-cyclohexyl-ethyl)-amide

3-Bromomethyl-2-(4-trifluoromethyl-phenyl)-quinoline-4-carboxylic acid ((S)-1-cyclohexyl-ethyl)-amide was prepared starting from isatine and (4-trifluoromethyl)propiophenone following the procedures described in Description 24-26.

### EXAMPLE 1

### 3-[1,4′]Bipiperidinyl-1′-ylmethyl-2-phenyl-quinoline-4-carboxylic Acid 3-hydroxy-benzylamide

0.54 g (1 mmol) of crude trihydrochloride of 3-[1,4′]bipiperidinyl-1′-ylmethyl-2-phenyl -quinoline-4-carboxylic acid (compound of description 2), 0.57 g (1.5 mmol) of HBTU and 690 microlitre of triethylamine were dissolved in 12 ml anhydrous THF. A solution of 0.15 g (1.2 mmol) of 3-hydroxybenzylamine (RN 73604-31-6) in 7 ml of methylene chloride was added and the mixture was stirred 15 h at room temperature. The solvent was evaporated in vacuo to dryness and the residue was taken up with AcOEt and washed with water. The organic phase was dried over MgSO₄ and concentrated to dryness. The residue was submitted to flash chromatography (silica gel, CH₂Cl₂/MeOH/NH₄OH: 94/6/0.6) and crystallisation in diisopropyl ether afforded 110 mg (yield 20.6%) of the title compound as beige crystals.

C₃₄H₃₈N₄O₂

MW=534.70

### EXAMPLE 2

### 3-[1,4′]Bipiperidinyl-1′-ylmethyl-2-phenyl-quinoline-4-carboxylic Acid Benzylamide

Prepared from 3-[1,4′]Bipiperidinyl-1′-ylmethyl-2-phenyl-quinoline-4-carboxylic acid dihydrochloride (compound of Description 2) and benzylamine following the procedure of Example 1.

C₃₄H₃₈N₄₀

MW=518.70

### EXAMPLE 3

### 3-[1,4′]Bipiperidinyl-1′-ylmethyl-2-phenyl-quinoline-4-carboxylic Acid ((S)-2-hydroxy-1-phenyl-ethyl)-amide

Prepared from 3-[1,4′]Bipiperidinyl-1′-ylmethyl-2-phenyl-quinoline-4-carboxylic acid dihydrochloride (compound of Description 2) and 1-phenylethanolamine following the procedure of Example1.

C₃₅H₄₀N₄O₂

MW=548.73

### EXAMPLE 4

### 3-[1,4′]Bipiperidinyl-1′-ylmethyl-2-phenyl-quinoline-4-carboxylic acid ((1S,2R)-2-hydroxy-1-methyl-2-phenyl-ethyl)-amide

Prepared from 3-[1,4′]Bipiperidinyl-1′-ylmethyl-2-phenyl-quinoline-4-carboxylic acid dihydrochloride (compound of Description 2) and 3-hydroxy-2-methyl-3-phenylpropylamine following the procedure of Example1.

C₃₆H₄₂N₄O₂

MW=562.75

### EXAMPLE 5

### 2-Phenyl-3-(4-piperazin-1-yl-piperidin-1-ylmethy)-quinoline-4-carboxylic Acid ((S)-1-phenyl-propyl)-amide

A mixture of 66 mg (0.085 mmol) of 4{1-[2-phenyl-4((S)-1-phenyl -propylcarbamoyl)-quinolin-3-ylmethyl]-piperidin-4-yl}-piperazine-1-carboxylic acid 9H-fluoren-9-ylmethyl ester (compound of Description 7), 0.013 ml of piperidine and 2 ml of acetonitrile was stirred at room temperature for 26 h. After concentration the residue was purified by flash chromatography (silica gel, first CH₂Cl₂/MeOH: 95/5 then CH₂Cl₂/MeOH/NH₄OH: 90/9/1) to afford 26 mg (yield 55%) of the title compound as a beige solid.

C₃₅H₄₁N₅O

MW=547.74

### EXAMPLE 6

### 3-(4-Amino-piperidin-1-ylmethyl)-2-phenyl-quinoline-4-carboxylic Acid ((S)-1-cyclohexyl-ethyl)-amide

A mixture of 145 mg (0.21 mmol) of {1-[4-((S)-1-cyclohexyl-ethylcarbamoyl)-2-phenyl-quinolin-3-ylmethyl]-piperidin-4-yl}-carbamic acid 9H-fluoren-9-ylmethyl ester (compound of Description 10), 7 ml of DMF, 8 ml of CH₂Cl₂ and 27 mg (0.31 mmol) of piperidine was stirred at room temperature for 16 h. The mixture was concentrated to dryness in vacuo then purified by flash chromatography on silicagel (eluent, first CH₂Cl₂/MeOH: 95/5, then CH₂Cl₂/MeOH/NH₄OH: 99/10/1).

The desired fractions were concentrated and the residue was triturated with diisopropyl ether affording, after careful drying, 110 mg of the title compound as white crystals

C₃₀H₃₈N₄O

MW=470.66

### EXAMPLE 7

### 3-(4-Amino-piperidin-1-ylmethyl)-2-phenyl-quinoline-4-carboxylic Acid ((S)-1-phenyl-ethyl)-amide

The title compound was synthetized according to Description 10 and Example 6.

C₃₀H₃₂N₄O

MW=464.61

### EXAMPLE 8

### 3-(4-Amino-piperidin-1-ylmethyl)-2-phenyl-quinoline-4-carboxylic Acid ((S)-2-methyl-1-phenyl-propyl)-amide

The title compound was synthetized according to Description 10 and Example 6.

C₃₂H₃₆N₄O

MW=492.66

### EXAMPLE 9

### 3-[1,4′]Bipiperidinyl-1′-ylmethyl-2-phenyl-quinoline-4-carboxylic Acid ((S)-1-cyclopropyl-1-phenyl-methyl)-amide

The title compound was synthesized starting from compound of Description 2 and (S) 1-cyclopropyl-1-phenylmethylamina following the procedure of Example 1

C₃₇H₄₂N₄O

MW=558.77

### EXAMPLE 10

### 3-[1,4′]Bipiperidinyl-1′-ylmethyl-2-phenyl-quinoline-4-carboxylic Acid ((R)-1-cyclopropyl-1-phenyl-methyl)-amide

The title compound was synthesized starting from compound of Description 2 and (R) 1-cyclopropyl-1-phenylmethylamina following the procedure of Example 1

C₃₇H₄₂N₄O

MW=558.77

### EXAMPLE 11

### 3-(2-Oxo-[1,4′]bipiperidinyl-1′-ylmethyl)-2-phenyl-quinoline-4-carboxylic Acid ((S)-1-phenyl-ethyl)-amide

A mixture of 0.5 g (1.1 mmol) of crude 3-(2-oxo-[1,4′]bipiperidinyl-1′-ylmethyl)-2-phenyl-quinoline-4-carboxylic acid (compound of Description 13), 0.44 g (4.4 mmol) of triethylamine, 0.65 g (1.65 mmol) of HBTU, 0.16 g (1.32 mmol) of(S)-(−)-1-phenyl propylamine, 10 ml of TBF and 10 ml of methylene chloride stabilised with amylene was stirred at room temperature for 18 h. The solvent was concentrated and the residue dissolved in AcOEt. The organic phase was washed with a 0.5 N NaOH solution, then with water and dried over MgSO₄. After concentration the residue was purified by flash chromatography (silica gel CH₂Cl₂/MeOH: 95/5) to afford 0.360 g (yield 57%) of the title compound as a white solid.

C₃₅H₃₈N₄O₂

MW=546.71

### EXAMPLE 12

### 3-[4-(2-Oxo-pyrrolidin-1-yl)-piperidin-1-ylmethyl]-2-phenyl -quinoline-4-carboxylic Acid ((S)-1-phenyl-ethyl)-amide

Applying the procedure of Example 1 to 0.640 g of crude 3-[4-(2-oxo-pyrrolidin-1-yl) -piperidin-1-ylmethyl]-2-phenyl-quinoline-4-carboxylic acid (compound of Description 14) afforded after purification 0.38 g of the title compound as a beige solid.

C₃₄H₃₆N₄O₂

MW=532.68

### EXAMPLE 13

### 1′-[4-((S)-1-Cyclohexyl-ethylcarbamoyl)-2-phenyl-quinolin-3-ylmethyl]-[1,4′]bipiperidinyl-3-carboxylic Acid Ethyl Ester

A mixture of 0.342 g (0.51 mmol) of 1′-(4-carboxy-2-phenyl-quinolin-3-ylmethyl)-[1,4′]bipiperidinyl-3-carboxylic acid ethyl ester (compound of Description 18), 350 microliters (2.5 mmol) triethylamine, 290 mg (0.76 mmol) HBTU, 8 ml anhydrous THF, 112 microliters (0.76 mmol) (S)-(+)-1-cyclohexylethylamine and 5 ml methylene chloride was stirred 16 h at room temperature. The mixture was concentrated in vacuo, the residue was dissolved in ethyl acetate and the organic phase washed with water.

After drying over MgSO₄ the solvent was concentrated and the residue purified by flash chromatography over 35 g silicagel (eluent: CH₂Cl₂/MeOH: 95/5) affording 290 mg of a crude compound.

A new chromatography over 40 g silicagel with the same eluent afforded 0.09 g of pure title compound.

C₃₈H₅₀N₄O₃

MW=610.84

### EXAMPLE 14

### 1′-[4-((S)-1-Cyclohexyl-ethylcarbamoyl)-2-phenyl-quinolin-3-ylmethyl]-[1,4′]bipiperidinyl-3-carboxylic Acid

A mixture of 0.19 g (0.3 mmol) of 1′-[4-((S)-1-cyclohexyl-ethylcarbamoyl)-2-phenyl -quinolin-3-ylmethyl]-[1,4′]bipiperidinyl-3-carboxylic acid ethyl ester (compound of Example 13) 3 ml of ethanol and 620 microliters of aqueous 1 N lithium hydroxide were stirred at room temperature for 5 h. A TLC confirming that the reaction was not complete, 200 microliters of LiOH were added and the mixture stirred for 15 additional hours. After concentration of the ethanol the mixture was dissolved in water and acidified with a saturated solution of KHSO₄. An attempt to extract the compound with methylene chloride having failed, the mixture was concentrated to dryness and the residue was purified by flash chromatography on 25 g silicagel (eluent: CH₂Cl₂/MeOH/NH₄OH: 9/1/0.1) yielding 0.13 g (74%) of the title compound.

C₃₆H₄₆N₄O₃

MW=582.78

### EXAMPLE 15

### 3-[1,4′]Bipiperidinyl-1′-ylmethyl-7-hydroxy-2-phenyl-quinoline-4-carboxylic Acid ((S)-1-cyclohexyl-ethyl)-amide

Prepared from 3-[1,4′]Bipiperidinyl-1′-ylmethyl-8-bromo-7-hydroxy-2-phenyl -quinoline-4-carboxylic acid hydrochloride (compound of Description 22) following the procedure of Description 23

C₃₅H₄₆N₄O₂

MW=554.77

### EXAMPLE 16

### [3-[1,4′]Bipiperidinyl-1′-ylmethyl-2-phenyl-4-((S)-1-phenyl -propylcarbamoyl)-quinolin-7-yloxy]-acetic Acid Ethyl Ester

1.0 g (1.78 mmol) of 3-[1,4′]Bipiperidinyl-1′-ylmethyl-7-hydroxy-2-phenyl-quinoline-4-carboxylic acid ((S)-1-phenyl-propyl)-amide (compound of Description 23), 0.62 g (4.45 mmol) of K₂CO₃ and a catalytic amount of potassium iodide were suspended in 20 ml of dry THF. 0.3 ml (2.67 mmol) of ethyl bromoacetate were added and the slurry was stirred at 60° C. for 10 hours, cooled to room temperature and evaporated to dryness. The residue was taken up with H₂O and extracted three times with EtOAc. The organic phases, collected together, were dried over Na₂SO₄ and evaporated in vacuo to dryness. The residue was purified by flash chromatography over silicagel (eluent EtOAc/MeOH/NH₄OH: 95/5/05). The crude compound was triturated in Et2O affording 0.81 g of the title compound.

C₄₀H₄₈N₄O₄.

MW=648.84

### EXAMPLE 17

### [3-[1,4′]Bipiperidinyl-1′-ylmethyl-2-phenyl-4((S)-1-phenyl -propylcarbamoyl)-quinolin-7-yloxy]-acetic Acid Dihydrochloride

0.3 g (0.46 mmol) of [3-[1,4′]bipiperidinyl-1′-ylmethyl-2-phenyl-4-((S)-1-phenyl -propylcarbamoyl)-quinolin-7-yloxy]-acetic acid ethyl ester (compound of Example 16) were suspended in 15 ml of 20% HCl and the mixture was refluxed for 4 hours. After cooling the solvent was removed in vacuo and the crude compound was triturated in Et2O affording 0.25 g of the title compound as a dark powder.

C₃₈H₄₄N₄O₄.2HCl

MW=693.80

### EXAMPLE 18

### 3-[1,4′]Bipiperidinyl-1′-ylmethyl-7-hydroxy-2-phenyl-quin line-4-carboxylic Acid ((S)-1-phenyl-ethyl)-amide

Prepared from 3-[1,4′]Bipiperidinyl-1′-ylmethyl-8-bromo-7-hydroxy-2-phenyl -quinoline-4-carboxylic acid hydrobromide (compound of Description 22) following the procedure of Description 23.

C₃₅H₄₀N₄O₂

MW=548.73

### EXAMPLE 19

### 3-[1,4′]Bipiperidinyl-1′-ylmethyl-7-(2-hydroxy-ethoxy)-2-phenyl -quinoline-4-carboxylic acid ((S)-1-phenyl-propyl)-amide

0.3 g (0.35 mmol) 3-[1,4′]bipiperidinyl-1′-ylmethyl-2-phenyl-4-((S)-1-phenyl -propylcarbamoyl)-quinolin-7-yloxy]-acetic acid dihydrochloride (compound of Example 17) were dissolved in 25 ml of tert-butanol, 0.25 g of sodiumborhydride were added and the mixture was refluxed for 6 h. After cooling, 10 ml of 6 N HCl solution were added dropwise. The solution was extracted with ethyl ether, basified with 1 N NaOH to pH=12 and extracted three times with EtOAc. The organic phases collected togheter and dried over Na₂SO₄ were evaporated in vacuo to dryness. The residue was purified by flash chromatography over silicagel (eluent EtOAc/MeOH/NH₄OH: 95/5/05). The crude compound was triturated in isopropyl ether affording 0.08 g of the title compound as a slightly yellow powder.

C₃₈H₄₆N₄O₃.

MW=648.84

### EXAMPLE 20

### 3-[1,4′]Bipiperidinyl-1′-ylmethyl-7-carbamoylmethoxy-2-phenyl -quinoline-4-carboxylic Acid ((S)-1-phenyl-propyl)-amide Dihydrochloride

0.28 g (0.5 mmol) of 3-[1,4′]Bipiperidinyl-1′-ylmethyl-7-hydroxy-2-phenyl-quinoline-4-carboxylic acid ((S)-1-phenyl-propyl)-amide (compound of Description 23) 0.21 g (1.5 mmol) of K₂CO₃ and a catalytic amount of potassium iodide were suspended in 10 ml of CH₃CN. 0.1 ml (0.75 mmol) of bromoacetamide were added and the slurry was stirred at 50° C. for 6 hours, cooled to room temperature and evaporated to dryness. The residue was taken up with EtOAc and washed with H₂O, dried over Na₂SO₄ and evaporated in vacuo to dryness. The residue was purified by flash chromatography over silicagel (eluent EtOAc/MeOH₄OH: 95/5/05). The crude compound was dissolved in Me₂CO and treated with a solution of HCl in diethyl ether. The slurry was evaporated in vacuo to dryness and the residue was triturated in Et₂O, filtered and dried in vacuo at 40° C. affording 0.1 g of the title compound as a white powder.

C₃₈H₄₅N₅O₃.

MW=619.82

### EXAMPLE 21

### 3-[1,4′]Bipiperidinyl-1′-ylmethyl-7-hydroxy-2-phenyl-quinoline-4-carboxylic Acid ((S)-2-methyl-1-phenyl-propyl)-amide

Prepared from 3-[1,4′]Bipiperidinyl-1′-ylmethyl-8-bromo-7-hydroxy-2-phenyl -quinoline-4-carboxylic acid hydrobromide (compound of Description 22) following the procedure of Description 23, affording the title compound as a yellowish powder.

C₃₇H₄₄N₄O₂

MW=576.78

### EXAMPLE 22

### 3-[1,4′]Bipiperidinyl-1′-ylmethyl-7-methoxy-2-phenyl-quinoline-4-carboxylic Acid ((S)-2-methyl-1-phenyl-propyl)-amide

Prepared from 3-[1,4′]Bipiperidinyl-1′-ylmethyl-8-bromo-7-methoxy-2-phenyl -quinoline-4-carboxylic acid hydrochloride (compound of Description 21) following the procedure of Description 23, affording the title compound as a white powder.

C₃₈H₄₆N₄O₂

MW=590.81

### EXAMPLE 23

### 3-[1,4′]Bipiperidinyl-1′-ylmethyl-2-phenyl-quinoline-4-carboxylic acid cyclohexylamide

Prepared from 3-[1,4′]Bipiperidinyl-1′-ylmethyl-2-phenyl-quinoline-4-carboxylic acid dihydrochloride (compound of Description 2) following the procedure of Description 10.

C₃₃H₄₂N₄O

MW=510.72

### EXAMPLE 24

### 3-[1,4′]Bipiperidinyl-1′-ylmethyl-7-chloro-2-phenyl-quinoline-4-carboxylic acid ((S)-1-cyclohexyl-ethyl)-amide

A solution of 3-bromomethyl-7-chloro-2-phenyl-quinoline-4-carboxylic acid ((S)-1-cyclohexyl-ethyl)-amide (0.2 g, 0.41 mmol), compound prepared as in Description 26, 4-piperidino-piperidine (85 mg, 0.5 mmol) and ethyldiisopropylamine (165 mg, 1.28 mmol,) in CH₂Cl₂ (15 ml) was refluxed for 3 h. The organic phase was washed with water and then dried over Na₂SO₄. After evaporating to dryness, the residue was triturated with iPrO₂ to obtain 17 mg of the title compound as beige crystals

C₃₅H₄₅FN₄O

MW=573.22

### EXAMPLE 25

### 3-[1,4′]Bipiperidinyl-1′-ylmethyl-7-fluoro-2-phenyl-quinoline-4-carboxylic Acid ((S)-1-cyclohexyl-ethyl)-amide

The title compound was obtained by reacting 3-bromomethyl-7-fluoro-2-phenyl -quinoline-4-carboxylic acid ((S)-1-cyclohexyl-ethyl)-amide, prepared as in Description 27, with 4-piperidino-piperidine following the procedure described in Example 24.

C₃₅H₄₅FN₄O

MW=556.77

### EXAMPLE 26

### 3-[1,4′]Bipiperidinyl-1′-ylmethyl-8-fluoro-2-phenyl-quinoline-4-carboxylic Acid ((S)-1-cyclohexyl-ethyl)-amide

The title compound was obtained by reacting 3-bromomethyl-8-fluoro-2-phenyl -quinoline-4-carboxylic acid ((S)-1-cyclohexyl-ethyl)-amide, prepared as in Description 28, with 4-piperidino-piperidine following the procedure described in Example 24.

C₃₅H₄₅FN₄O

MW=556.770

### EXAMPLE 27

### 3-[1,4′]Bipiperidinyl-1-ylmethyl-2-thiophen-2-yl-quinoline-4-carboxylic Acid ((S)-1-cyclohexyl-ethyl)-amide

The title compound was obtained by reacting 3-bromomethyl-2-thiophen-2-yl -quinoline-4-carboxylic acid ((S)-1-cyclohexyl-ethyl)-amide, prepared as in Description 29, with 4-piperido-piperidine following the procedure described in Example 24.

C₃₃H₄₄N₄OS

MW=544.800

### EXAMPLE 28

### 3-[1,4′]Bipiperidinyl-1′-ylmethyl-6-fluoro-2-phenyl-quinoline-4-carboxylic Acid ((S)-1-cyclohexyl-ethyl)-amide

The title compound was obtained by reacting 3-bromomethyl-6-fluoro-2-phenyl-quinoline 4 carboxylic acid ((S)-1-cyclohexyl-ethyl)-amide, prepared as in Description 30, with 4-piperidino-piperidine following the procedure described in Example 24

C₃₅H₄₅FN₄O

MW=556.766

### EXAMPLE 29

### 3-[1,4′]Bipiperidinyl-1′-ylmethyl-2-(4-fluoro-phenyl)-quinoline-4-carboxylic Acid ((S)-1-cyclohexyl-ethyl)-amide

The title compound was obtained by reacting 3-bromomethyl-2-(4-fluorophenyl) -quinoline-4-carboxylic acid ((S)-1-cyclohexyl-ethyl)-amide, prepared as in Description 32, with 4-piperidino-piperidine following the procedure described in Example 24.

C₃₅H₄₅FN₄O

MW=556.766

### EXAMPLE 30

### 3-[1,4′]Bipiperidinyl-1′-ylmethyl-2-(4-trifluoromethyl-phenyl) -quinoline-4-carboxylic Acid ((S)-1-cyclohexyl-ethyl)-amide

The title compound was obtained by reacting 3-bromomethyl-2-(4-tifluoromethylphenyl)-quinoline-4-carboxylic acid ((S)-1-cyclohexyl-ethyl)-amide, prepared as in Description 33, with 4-piperidino-piperidine following the procedure described in Example 24

C₃₆H₄₅F₃N₄₀

MW=606.777

### EXAMPLE 31

### 3-[1,4′]Bipiperidinyl-1′-ylmethyl-2-(2-fluoro-phenyl)-quinoline-4-carboxylic Acid ((S)-1-cyclohexyl-ethyl)-amide

The title compound was obtained by reacting 3-bromomethyl-2-(2-fluorophenyl) -quinoline-4-carboxylic acid ((S)-1-cyclohexyl-ethyl)-amide, prepared as in Description 31, with 4-piperidino-piperidine following the procedure described in Example 24.

### EXAMPLE 32

### 3-[1,4′]Bipiperidinyl-1′-ylmethyl-2-phenyl-6-trifluoromethyl-quinoline-4-carboxylic Acid ((S)-1-cyclohexyl-ethyl)-amide

The title compound was obtained by reacting 3-bromomethyl-2-phenyl-6-trifluorom ethyl-quinoline-4-carboxylic acid ((S)-1-cyclohexyl-ethyl)-amide, prepared as in Description 24-26 starting from 5-trifluoromethylisatin (Tetrahedron Letters, 35, 7303, 1994), with 4-piperidino-piperidine following the procedure described in Example 24

C₃₆H₄₅F₃N₄O

MW=606.78

### EXAMPLE 33

### 3-[1,4′]Bipiperidinyl-1′-ylmethyl-2-phenyl-7-trifluoromethyl-quinoline-4-carboxylic Acid ((S)-1-cyclohexyl-ethyl)-amide

The title compound was obtained by reacting 3-bromomethyl-2-phenyl-7-trifluorom ethyl-quinoline-4-carboxylic acid ((S)-1-cyclohexyl-ethyl)-amide, prepared as in Description 24-26 staring from 6-trifluoromethylisatin (Tetrahedron Letters, 35, 7303, 1994), with 4-piperidino-piperidine following the procedure described in Example 24

C₃₆H₄₅F₃N₄₀

MW=606.78

TABLE 1

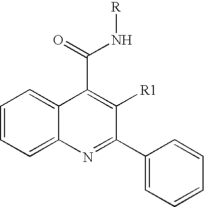

| | | | | Molecular | Melting Point | |
|---|---|---|---|---|---|---|
| Ex. | R | R₁ | Molecular Formula | Weight | (° C.) | [a]_{D}²⁰ |
| 1 | 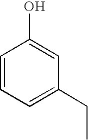 | 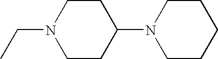 | C₃₄H₃₈N₄O₂ | 534.70 | 135-137 | — |
| 2 | 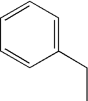 | 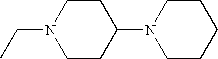 | C₃₄H₃₈N₄O | 518.70 | 160 | — |
| 3 | 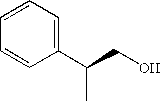 | 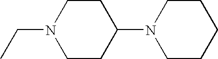 | C₃₅H_{4O}N₄O₂ | 548.73 | 194-195 | — |
| 4 | 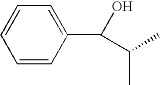 | 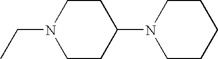 | C₃₆H₄₂N₄O₂ | 562.75 | — | — |
| 5 | 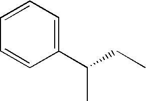 | 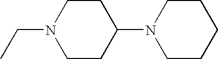 | C₃₅H₄₁N₅O | 547.74 | — | — |
| 6 | 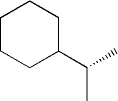 | 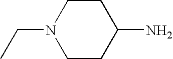 | C₃₀H₃₈N₄O | 470.66 | 140-145 | — |
| 7 | 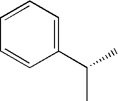 | 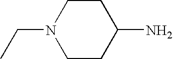 | C₃₀H₃₂N₄O | 464.61 | 118-119 | — |
| 8 | 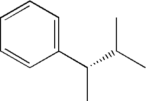 | 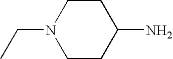 | C₃₂H₃₆N₄O | 492.66 | 111-112 | — |
| 9 | 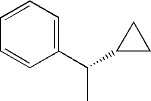 | 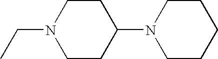 | C₃₇H₄₂N₄O | 558.77 | 92-93 | — |
| 10 | 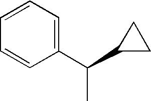 | 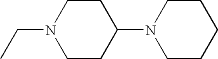 | C₃₇H₄₂N₄O | 558.77 | 85-86 | — |
| 11 | 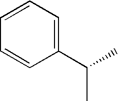 | 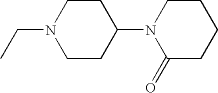 | C₃₅H₃₈N₄O₂ | 546.71 | 124-125 | — |
| 12 | 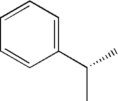 | 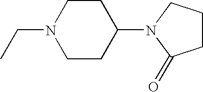 | C₃₄H₃₆N₄O₂ | 532.68 | 120-125 | — |
| 13 | 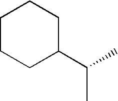 | 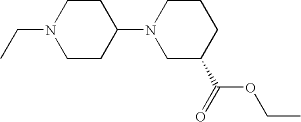 | C₃₈H₅₀N₄O₃ | 610.84 | 104-105 | — |
| 14 | 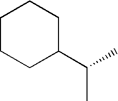 | 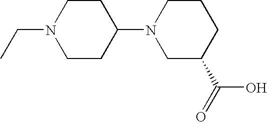 | C₃₆H₄₆N₄O₃ | 582.78 | 160-165 | — |
| 15 | 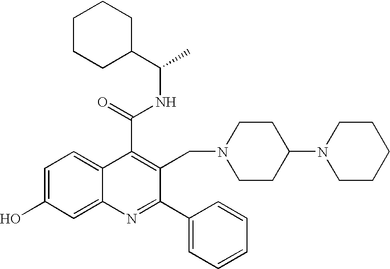 | | C₃₅H₄₆N₄O₂ | 554.77 | 177.9-178 | +19.89 (c = 0.38, MeOH) |
| 16 | 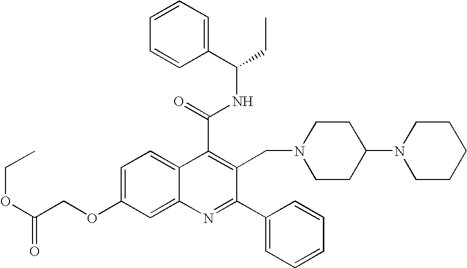 | | C₄₀H₄₈N₄O₄ | 648.84 | 101-104 | −32.07 (c = 0.44, MeOH) |
| 17 | 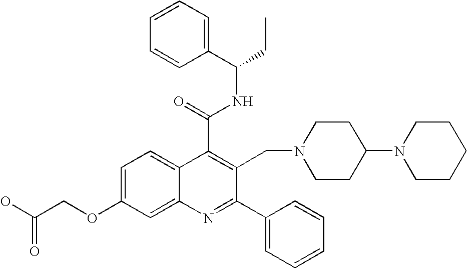 | | C₃₈H₄₄N₄O₄.2HCl | 693.80 | 196 dec | +5.25 (c = 0.35, MeOH) |
| 18 | 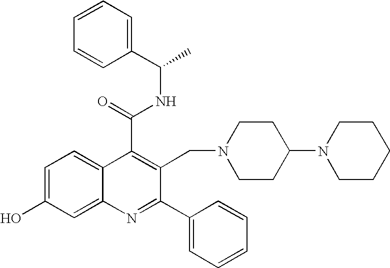 | | C₃₅H₄₀N₄O₂ | 548.73 | 99.6-99.7 | −28.56 (c = 0.33, MeOH) |
| 19 | 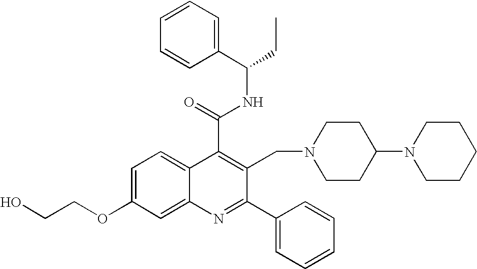 | | C₃₈H₄₆N₄O₃ | 648.84 | 118-122 | −36.18 (c = 0.27, MeOH) |
| 20 | 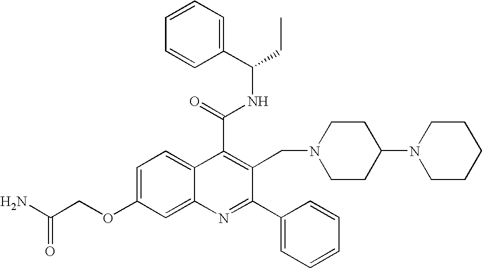 | | C₃₈H₄₅N₅O₃ | 619.82 | 218-220 | +3.18 (c = 0.34, MeOH) |
| 21 | 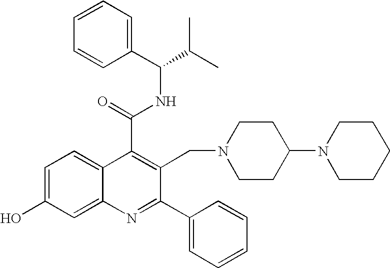 | | C₃₇H₄₄N₄O₂ | 576.78 | 200-204 (dec) | −61.6 (c = 0.22, MeOH) |
| 22 | 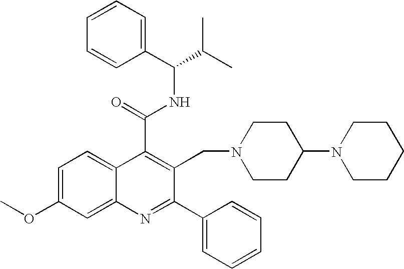 | | C₃₈H₄₆N₄O₂ | 590.81 | 113-116 | −50.3 (c = 0.26, MeOH) |
| 23 | 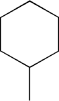 | 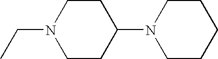 | C₃₃H₄₂N₄O | 510.72 | 205.6-205.7 | — |

| | | | | | |
|---|---|---|---|---|---|
| 24 | 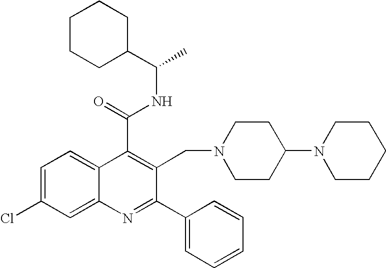 | C₃₅H₄₅ClN₄O | 573.22 | 152-154 | — |
| 25 | 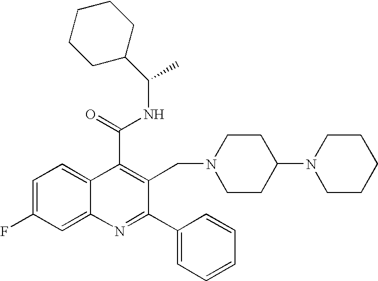 | C₃₅H₄₅FN₄O | 556.77 | 161-163 | +15.24 (c = 0.3, MeOH) |
| 26 | 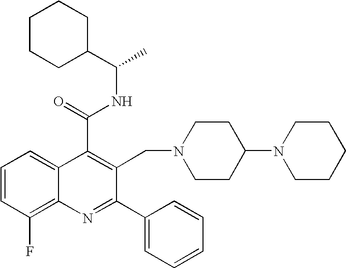 | C₃₅H₄₅FN₄O | 556.770 | 113 | +12.71 (c = 0.1, MeOH) |
| 27 | 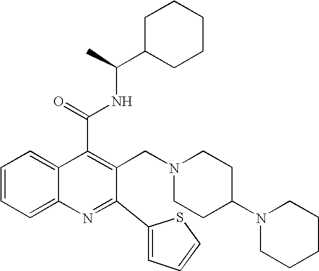 | C₃₃H₄₄N₄OS | 544.800 | 165-166 | +13.2 (c = 0.2, MeOH) |
| 28 | 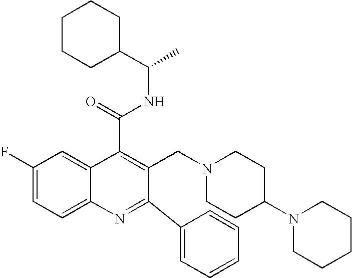 | C₃₅H₄₅FN₄O | 556.766 | 174-175 | — |
| 29 | 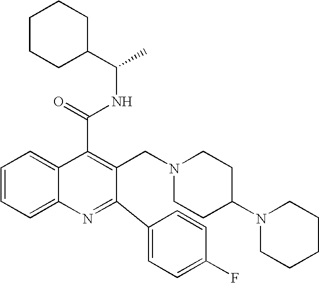 | C₃₅H₄₅FN₄O | 556.766 | 151-153 | +11.69 (c = 0.5, MeOH) |
| 30 | 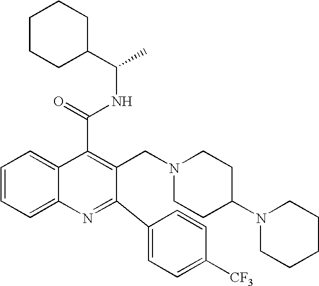 | C₃₆H₄₅F₃N₄O | 606.777 | 138-140 | +8.9 (c = 0.2, MeOH) |
| 31 | 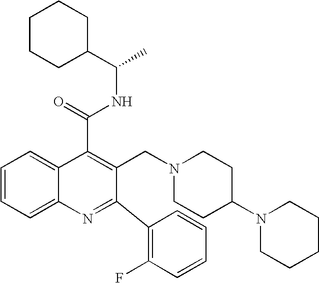 | C₃₅H₄₅FN₄O | 556.766 | — | — |
| 32 | 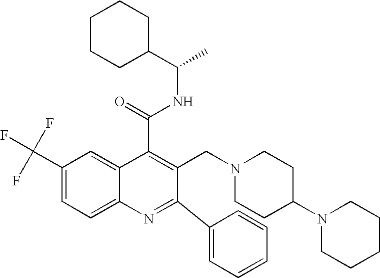 | C₃₆H₄₅F₃N₄O | 606.78 | — | — |
| 33 | 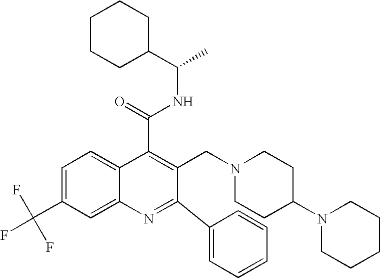 | C₃₆H₄₅F₃N₄O | 606.78 | — | — |

TABLE 2
¹H NMR data of compounds of Examples of Table 1

| Ex. | ¹H NMR (Solvent) δ |
|---|---|
| 1 | (CDCl₃): 1.09-1.80(10H); 1.96(m, 1H); 2.31-2.64(m, 9H); 3.68(s, 2H); 4.70(d, 2H); 6.77(m, 1Har); 6.91 |
| | (d, 1H); 7.03(s, 1Har); 7.19(t, 1Har); 7.35-7.51(m, 5Har); 7.58(td, 1Har); 7.73(td, 1Har); 8.07-8.19 |
| | (2Har); 8.63(m br, 1H) |
| 2 | (CDCl₃): 0.81-1.08(m, 2H); 1.21-2.17(m, 11H); 2.19-2.45(m, 6H); 3.61(s, 2H); 4.75(d, 2H); 7.26-7.52 |
| | (m, 10Har); 7.61(td, 1Har); 7.75(td, 1Har); 8.13(dd, 1Har); 8.21(dd, 1Har); 9.55(br, 1H) |
| 3 | (CDCl₃): 0.77-2.15(m, 12H); 2.31(m, 4H); 2.50(m, 1H); 2.69(m, 1H); 3.65-3.80(m, 2H); 4.07(m, 2H); |
| | 5.47(m, 1H); 7.28-7.52(m, 10Har); 7.57(td, 1Har); 7.74(td, 1Har); 8.04-8.19(m, 2Har); 9.18(d br, 1H) |
| 4 | (CDCl₃): 1.05-1.76(m, 10H); 1.23(d, 3H); 1.85-2.15(m, 2H); 2.22-2.48(m, 5H); 2.62(m, 1H); 3.66(m, |
| | 2H); 4.63(m, 1H); 5.07(d, 1H); 7.20-7.52(11Har); 7.57(td, 1Har); 7.74(td, 1Har); 8.11(m, 2Har); 8.75(d |
| | br, 1H) |
| 5 | (CDCl₃): 0.87-1.15(m, 2H); 1.04(t, 3H); 1.29-1.66(m, 4H); 1.73-2.19(m, 5H); 2.41(m, 4H); 2.49(m, 1H); |
| | 2.89(m, 4H); 3.57(s, 2H); 5.32(m, 1H); 7.27-7.50(m, 10Har); 7.55(t, 1Har); 7.73(td, 1Har); 8.06(d, |
| | 1Har); 8.12(dd, 1Har); 8.75(d br, 1H) |
| 6 | (CDCl₃): 0.92-2.05(m, 19H); 1.29(d, 3H); 2.35-2.77(3H); 3.71(dd, 2H); 4.27(m, 1H); 7.46(m, 5Har); |
| | 7.58(td, 1Har); 7.73(td, 1Har); 8.11(m, 2Har); 8.20(br, 1H) |
| 7 | (CDCl₃): 0.55-1.00(m, 2H); 1.26-1.80(m, 6H); 1.71(d, 3H); 2.10(m, 1H); 2.43(m, 2H); 3.62(s, 2H); 5.56 |
| | (m, 1H); 7.20-7.65(m, 1Har); 7.74(td, 1Har); 8.05-8.18(m, 2Har); 9.12(d br, 1H) |
| 8 | (CDCl₃): 0.78-1.10(m, 2H); 0.94(d, 3H); 1.17(d, 3H); 1.37-1.79(m, 6H); 2.06(m, 1H); 2.18-2.52(m, 3H); |
| | 3.52(s, 2H); 5.15(m, 1H); 7.20-7.60(m, 11Har); 7.72(td, 1Har); 8.00(m, 1Har); 8.11(dd, 1Har); 8.35(d |
| | br, 1H) |
| 9 | (CDCl₃): 0.45-1.78(m, 17H); 1.97(m, 2H); 2.29(m, 4H); 2.65(m, 1H); 3.64(m, 2H); 4.95(m, 1H); 7.20-7.67 |
| | (m, 11Har); 7.74(td, 1Har); 8.13(dd, 2Har); 9.74(br, 1H) |
| 10 | (CDCl₃): 0.45-1.78(m, 17H); 1.97(m, 2H); 2.29(m, 4H); 2.65(m, 1H); 3.64(m, 2H); 4.95(m, 1H); 7.20-7.67 |
| | (m, 11Har); 7.74(td, 1Har); 8.13(dd, 2Har); 9.74(br, 1H) |
| 11 | (CDCl₃): 0.70-1.40(m, 4H); 1.55-1.87(m, 6H); 1.75(d, 3H); 2.15-2.40(m, 3H); 2.59(m, 1H); 2.74(m, |
| | 2H); 3.66(s, 2H); 4.06(m, 1H); 7.20-7.54(m, 10Har); 7.59(td, 1Har); 7.74(td, 1Har); 8.05-8.20(m, 2Har); |
| | 9.20(br, 1H) |
| 12 | (CDCl₃): 0.75-1.48(m, 4H); 1.55-2.00(m, 5H); 1.74(d, 3H); 2.23(m, 1H); 2.30(t, 2H); 2.58(m, 1H); 2.01 |
| | (t, 2H); 3.66(m, 2H); 5.54(m, 1H); 7.20-7.53(m, 10Har); 7.58(td, 1Har); 7.75(td, 1Har); 8.13(d, 2Har); |
| | 9.02(d br, 1H) |
| 13 | (CDCl₃): 1.23(t, 3H); 1.30(d, 3H); 0.95-2.08(m, 20H); 2.10-3.08(m, 9H); 3.73(m, 2H); 4.06(q, 2H); 4.26 |
| | (m, 1H); 7.47(m, 5Har); 7.58(td, 1Har); 7.73(td, 1Har); 8.08(dd, 1Har); 8.13(dd, 1Har); 7.40-8.30(broad |
| | band, 1H) |
| 14 | (CDCl₃): 0.98-2.10(m, 24H); 2.36-3.13(m, 8H); 3.71(s, 2H); 4.26(m, 1H); 7.4(broad band, 2H); 7.47(m, |
| | 5Har); 7.59(td, 1Har); 7.74(td, 1Har); 8.02(dd, 1Har); 8.13(dd, 1Har) |
| 15 | (DMSO-d₆): 1.19(d, 3H); 1.56-1.03(m, 16H); 1.86-1.60(m, 7H); 1.99(tt, 1H); 2.35(m, 4H); 2.5(m, 2H); |
| | 3.05(s, 2H); 4.01(dq, 1H); 7.17(dd, 1H); 7.24(d, 1H); 7.47-7.38(m, 3H); 7.52(m, 2H); 7.69(d, 1H); 8.22 |
| | (d br, 1H); 9.84(s br, 1H) |
| 16 | (DMSO-d₆)(343K): 0.95(t, 3H); 0.95(t, 3H); 1.06(m, 2H); 1.24(t, 3H); 1.63-1.30(m, 10H); 1.97-1.80 |
| | (m, 3H); 2.33(m, 4H); 2.40(m, 2H); 3.44 and 3.39(ABq, 2H); 4.21(q, 2H); 4.92(s, 2H); 5.07(dt, 1H); |
| | 7.27(m, 2H); 7.37(dd, 2H); 7.54-7.41(m, 7H); 7.62(d, 1H); 8.84(d br, 1H) |
| 17 | (DMSO-d₆)as Na salt: 0.95(t, 3H); 1.24-1.09(m, 2H); 1.69-1.38(m, 10H); 1.99-1.75(m, 2H); 2.47-2.26 |
| | (m, 3H); 2.63(m, 4H); 3.44 and 3.39(ABq, 2H); 4.65(s, 2H); 5.07(dt, 1H); 7.54-7.20(m, 12H); 7.60(d, |
| | 1H); 8.89(d, 1H) |
| 18 | (DMSO-d₆): 1.14-1.03(m, 2H); 1.49-1.29(m, 9H); 1.53(d, 3H); 1.61(m, 2H); 2.33(m, 4H); 2.46(m, 2H); |
| | 3.42(s, 2H); 5.31(dt, 1H); 7.12(dd, 1H); 7.24(d, 1H); 7.52-7.27(m, 10H); 7.59(d, 1H); 8.83(d, 1H); 9.86 |
| | (s br, 1H) |
| 19 | (DMSO-d₆)(343K): 0.95(t, 3H); 1.06(m, 2H); 1.65-1.29(m, 10H); 1.97-1.76(m, 3H); 2.32(m, 4H); 2.41 |
| | (m, 2H); 3.41(m, 2H); 3.80(dt br, 2H); 4.18(t, 2H); 4.61(t br, 1H); 5.07(dt, 1H); 7.20(dd, 1H); 7.27(dd, |
| | 1H); 7.48-7.33(m, 8H); 7.53(m, 2H); 7.60(d, 1H);(d br, 1H) |
| 20 | (DMSO-d₆)(as a base 343K): 0.95(t, 3H); 1.05(m, 2H); 1.63-1.30(m, 10H); 2.00-1.75(m, 3H); 2.32(m, |
| | 4H); 2.40(m, 2H); 3.44 and 3.40(ABq, 2H); 4.60(s, 2H); 5.07(dt, 1H); 7.56-7.14(m, 4H); 7.61(d, 1H); |
| | 8.86(d, 1H) |
| 21 | (DMSO-d₆): 0.82(d, 3H); 1.01(m, 2H); 1.01(m, 2H); 1.08(d, 3H); 1.55-1.27(m, 10H); 1.92(m, 1H); 2.17-2.05 |
| | (m, 1H); 2.45-2.25(m, 6H); 3.36 and 3.30(ABq, 2H); 4.88(t, 1H); 7.06(dd, 1H); 7.53-7.23(m, 12H); |
| | 8.80(d, 1H);9.87(s br, 1H) |
| 22 | (DMSO-d₆): 0.82(d, 3H); 1.01(m, 2H); 1.07(d, 3H); 1.60-1.23(m, 10H); 1.84(m, 1H); 2.50-2.25(m, 6H); |
| | 2.1(m, 1H); 3.35(m, 2H); 3.92(s, 3H); 4.89(dd, 1H); 7.57-7.14(m, 13H); 8.83(d, 1H) |
| 23 | (DMSO-d₆): 1.82-1.05(m, 22H); 2.05-1.90(m, 3H); 2.32(m, 4H); 3.51(s, 2H); 3.90(m, 1H); 7.54-7.43(m, |
| | 5H); 7.64(dd, 1H); 7.77(dd, 1H); 7.85(d, 1H); 8.00(d, 1H); 8.55(d, 1H) |
| 24 | (DMSO-d₆, 333K): 8.37(d br, 1H); 8.05(d, 1H); 7.86(d, 1H); 7.67(dd, 1H); 7.57-7.40(m, 5H); 4.02(m, |
| | 1H); 3.55(s, 2H); 2.50(m, 2H); 2.35(m, 4H); 1.99(tt, 1H); 1.86-1.57(m, 8H); 1.53-1.01(m, 15H); 1.91(d, |
| | 3H) |
| 25 | (DMSO-d₆, 343K): 8.29(d br, 1H); 7.91(d, 1H); 7.71(dd, 1H); 7.58-7.49(m, 3H); 7.49-7.40(m, 3H); 4.03 |
| | (m, 1H); 2.50(m, 2H); 3.56(s, 2H); 2.34(m, 4H); 1.98(tt, 1H); 1.86-1.60(m, 6H); 1.5-1.04(m, 17H); 1.20 |
| | (d, 3H) |
| 26 | (DMSO-d₆, 343K): 8.29(d br, 1H); 7.66(dd, 1H); 7.61(dd, 1H); 7.58-7.52(m, 3H); 7.51-7.42(m, 3H); |
| | 4.02(m, 1H); 3.58(s, 2H); 2.50(m, 2H); 2.37(m, 4H); 2.01(tt, 1H); 1.87-1.59(m, 6H); 1.54-1.05(m, |
| | 17H); 1.20(d, 3H) |
| 27 | (DMSO-d₆, 343K): 8.27(d br, 1H); 7.98(d, 1H); 7.93(d, 1H); 7.80(d, 1H); 7.75(dd, 1H); 7.67(d, 1H); |
| | 7.59(dd, 1H); 7.18(dd, 1H); 4.05(m, 1H); 3.73(s, 2H); 2.80(m, 2H); 2.40(m, 4H); 2.11(tt, 1H); 1.97(m, |
| | 2H); 1.85-1.09(m, 21H); 1.20(d, 3H) |
| 28 | (DMSO-d₆, 343K): 8.30(d br, 1H); 8.08(dd, 1H); 7.65(ddd, 1H); 7.56-7.51(m, 3H); 7.50-7.42(m, 3H); |
| | 4.04(m, 1H); 3.57(s, 2H); 2.50(m, 2H); 2.34(m, 4H); 1.99(tt, 1H); 1.86-1.62(m, 6H); 1.57-1.05(m, |
| | 17H); 1.20(d, 3H) |
| 29 | (DMSO-d₆, 343K): 8.27(d br, 1H); 8.01(d, 1H); 7.85(d, 1H); 7.75(dd, 1H); 7.62(m, 3H); 7.25(dd, |
| | 2H); 4.03(m, 1H); 3.55(s, 2H); 2.51(m, 2H); 2.35(m, 4H); 1.99(tt, 1H); 1.87-1.60(m, 6H); 1.53-1.06(m, |
| | 17H); 1.19(d, 3H) |
| 30 | (DMSO-d₆, 343K): 8.27(d br, 1H); 8.03(d, 1H); 7.86(d, 1H); 7.81-7.74(m, 5H); 7.65(dd, 1H); 4.05(m, |
| | 1H); 3.58(s, 2H); 2.49(m, 2H); 2.32(m, 4H); 1.98(tt, 1H); 1.89-1.61(m, 7H); 1.55-0.93(m, 16H); 1.20(d, |
| | 3H) |

TABLE 3
Mass Spectra data of compounds of Examples of Table 1

| | m/z (ESI POS; AQA; | m/z |
|---|---|---|
| | solvent: methanol/spray 3 kV/ | (EI+; TSQ 700; source |
| Ex. | skimmer: 20 V/probe 135 C) | 180° C.; 70 V; 200 uA) |
| 15 | 555 (MH+) | |
| 16 | 649 (MH+) | |
| 17 | 621 (MH+); 311 (MHH++) | |
| 18 | 549 (MH+) | |
| 19 | 607 (MH+) | |
| 20 | 620 (MH+) | |
| 21 | 577 (MH+) | |
| 22 | 591 (MH+) | |
| 23 | 511 (MH+); 256 (MHH++) | |
| 24 | | 572 (M+); 489; 167 |
| 25 | | 556 (M+); 402; 167 |
| 26 | | 556 (M+); 390; 167 |
| 27 | | 544 (M+); 378; 167 |
| 29 | | 556 (M+); 167 |
| 30 | | 167 |
| 31 | 557 (M+); 389; 300; 279; 169 | |

TABLE 4
Chemical names of parent compounds of Examples of Table 1
(names generated by Beilstein's Autonom)

| Example | Chemical name |
|---|---|
| 1 | 3-[1,4′]Bipiperidinyl-1′-ylmethyl-2-phenyl-quinoline-4-carboxylic acid 3-hydroxy-benzylamide |
| 2 | 3-[1,4′]Bipiperidinyl-1′-ylmethyl-2-phenyl-quinoline-4-carboxylic acid benzylamide |
| 3 | 3-[1,4′]Bipiperidinyl-1′-ylmethyl-2-phenyl-quinoline-4-carboxylic acid ((S)-2-hydroxy-1-phenyl-ethyl)-amide |
| 4 | 3-[1,4′]Bipiperidinyl-1′-ylmethyl-2-phenyl-quinoline-4-carboxylic acid ((1S, 2R)-2-hydroxy-1-methyl-2- |
| | phenyl-ethyl)-amide |
| 5 | 2-Phenyl-3-(4-piperazin-1-yl-piperidin-1-ylmethyl)-quinoline-4-carboxylic acid ((S)-1-phenyl-propyl)-amide |
| 6 | 3-(4-Amino-piperidin-1-ylmethyl)-2-phenyl-quinoline-4-carboxylic acid ((S)-1-cyclohexyl-ethyl)-amide |
| 7 | 3-(4-Amino-piperidin-1-ylmethyl)-2-phenyl-quinoline-4-carboxylic acid ((S)-1-phenyl-ethyl)-amide |
| 8 | 3-(4-Amino-piperidin-1-ylmethyl)-2-phenyl-quinoline-4-carboxylic acid ((S)-2-methyl-1-phenyl-propyl)- |
| | amide |
| 9 | 3-[1,4′]Bipiperidinyl-1′-ylmethyl-2-phenyl-quinoline-4-carboxylic acid ((S)-1-cyclopropyl-1-phenyl-methyl)- |
| | amide |
| 10 | 3-[1,4′]Bipiperidinyl-1′-ylmethyl-2-phenyl-quinoline-4-carboxylic acid ((R)-1-cyclopropyl-1-phenyl-methyl)- |
| | amide |
| 11 | 3-(2-Oxo-[1,4′]bipiperidinyl-1′-ylmethyl)-2-phenyl-quinoline-4-carboxylic acid ((S)-1-phenyl-ethyl)-amide |
| 12 | 3-[4-(2-Oxo-pyrrolidin-1-yl)-piperidin-1-ylmethyl]-2-phenyl-quinoline-4-carboxylic acid ((S)-1-phenyl- |
| | ethyl)-amide |
| 13 | 1′-[4-((S)-1-Cyclohexyl-ethylcarbamoyl)-2-phenyl-quinolin-3-ylmethyl]-[1,4′]bipiperidinyl-3-carboxylic acid |
| | ethyl ester |
| 14 | 1′-[4-((S)-1-Cyclohexyl-ethylcarbamoyl)-2-phenyl-quinolin-3-ylmethyl]-[1,4′]bipiperidinyl-3-carboxylic acid |
| 15 | 3-[1,4′]Bipiperidinyl-1′-ylmethyl-7-hydroxy-2-phenyl-quinoline-4-carboxylic acid ((S)-1-cyclohexyl-ethyl)- |
| | amide |
| 16 | [3-[1,4′]Bipiperidinyl-1′-ylmethyl-2-phenyl-4-((S)-1-phenyl-propylcarbamoyl)-quinolin-7-yloxy]-acetic acid |
| | ethyl ester |
| 17 | [3-[1,4′]Bipiperidinyl-1′-ylmethyl-2-phenyl-4-((S)-1-phenyl-propylcarbamoyl)-quinolin-7-yloxy]-acetic acid |
| | dihydrochloride |
| 18 | 3-[1,4′]Bipiperidinyl-1′-ylmethyl-7-hydroxy-2-phenyl-quinoline-4-carboxylic acid ((S)-1-phenyl-ethyl)-amide |
| 19 | 3-[1,4′]Bipiperidinyl-1′-ylmethyl-7-(2-hydroxy-ethoxy)-2-phenyl-quinoline-4-carboxylic acid ((S)-1-phenyl- |
| | propyl)-amide |
| 20 | 3-[1,4′]Bipiperidinyl-1′-ylmethyl-7-carbamoylmethoxy-2-phenyl-quinoline-4-carboxylic acid ((S)-1-phenyl- |
| | propyl)-amide dihydrochloride |
| 21 | 3-[1,4′]Bipiperidinyl-1′-ylmethyl-7-hydroxy-2-phenyl-quinoline-4-carboxylic acid ((S)-2-methyl-1-phenyl- |
| | propyl)-amide |
| 22 | 3-[1,4′]Bipiperidinyl-1′-ylmethyl-7-methoxy-2-phenyl-quinoline-4-carboxylic acid ((S)-2-methyl-1-phenyl- |
| | propyl)-amide |
| 23 | 3-[1,4′]Bipiperidinyl-1′-ylmethyl-2-phenyl-quinoline-4-carboxylic acid cyclohexylamide |
| 24 | 3-[1,4′]Bipiperidinyl-1′-ylmethyl-7-chloro-2-phenyl-quinoline-4-carboxylic acid ((S)-1-cyclohexyl-ethyl)- |
| | amide |
| 25 | 3-[1,4′]Bipiperidinyl-1′-ylmethyl-7-fluoro-2-phenyl-quinoline-4-carboxylic acid ((S)-1-cyclohexyl-ethyl)- |
| | amide |
| 26 | 3-[1,4′]Bipiperidinyl-1′-ylmethyl-8-fluoro-2-phenyl-quinoline-4-carboxylic acid ((S)-1-cyclohexyl-ethyl)- |
| | amide |
| 27 | 3-[1,4′]Bipiperidinyl-1′-ylmethyl-2-thiophen-2-yl-quinoline-4-carboxylic acid ((S)-1-cyclohexyl-ethyl)-amide |
| 28 | 3-[1,4′]Bipiperidinyl-1′-ylmethyl-6-fluoro-2-phenyl-quinoline-4-carboxylic acid ((S)-1-cyclohexyl-ethyl)- |
| | amide |
| 29 | 3-[1,4′]Bipiperidinyl-1′-ylmethyl-2-(4-fluoro-phenyl)-quinoline-4-carboxylic acid ((S)-1-cyclohexyl-ethyl)- |
| | amide |
| 30 | 3-[1,4′]Bipiperidinyl-1′-ylmethyl-2-(4-trifluoromethyl-phenyl)-quinoline-4-carboxylic acid ((S)-1-cyclohexyl- |
| | ethyl)-amide |
| 31 | 3-[1,4′]Bipiperidinyl-1′-ylmethyl-2-(2-fluoro-phenyl)-quinoline-4-carboxylic acid ((S)-1-cyclohexyl-ethyl)- |
| | amide |
| 32 | 3-[1,4′]Bipiperidinyl-1′-ylmethyl-2-phenyl-6-trifluoromethyl-quinoline-4-carboxylic acid ((S)-1-cyclohexyl- |
| | ethyl)-amide |
| 33 | 3-[1,4′]Bipiperidinyl-1′-ylmethyl-2-phenyl-7-trifluoromethyl-quinoline-4-carboxylic acid ((S)-1-cyclohexyl- |
| | ethyl)-amide |

1 A compound of formula (i) below or a pharmaceutically acceptable salt or hydrate thereof: 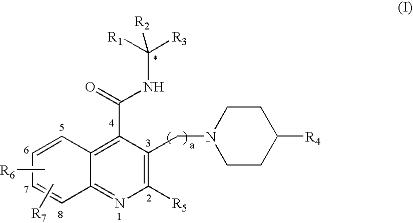
wherein:
R₁ is H or alkyl;
R₂ is —R₈R₉;
R₈ is a single bond or C₁₋₃ alkyl, optionally substituted one or more times by hydroxy;
R₉ is aryl or cycloalkyl or heteroaryl, optionally substituted one or more times by hydroxy, alkoxy, or alkoxyalkyl;
R₃ is H or alkyl or cycloalkyl or cycloalkylalkyl, optionally substituted one or more times by hydroxy or by one or more fluorines;
R₄ is —NR₁₀R₁₁;
R₁₀ and R₁₁ are independently selected from H or alkyl, or R₁₀ and R₁₁ together with the nitrogen atom to which they are attached form a saturated or unsaturated heterocyclic ring comprising 3-8 ring members, which heterocyclic ring is unsubstituted or is substituted one or more times by one or more substituents R₁₂;
R₁₂ is oxo or —R₁₃R₁₄R₁₅, wherein R₁₃ is a single bond or alkyl, R₁₄ is OC(O) or C(O)O, and R₁₅ is H or alkyl;
R₅ is an alkyl, cycloalkyl, cycloalkylalkyl, aryl, or single or fused ring aromatic heterocyclic group, which group is unsubstituted or is substituted one or more times by one or more substituents selected from halo such as fluoro, alkyl or haloalkyl such as fluoroalkyl;
R₆ represents H or up to three substituents independently selected from the list consisting of: alkyl, alkenyl, aryl, alkoxy or a hydroxylated derivative thereof, hydroxy, halogen, nitro, cyano, carboxy, carboxamido, sulphonamido, alkoxycarbonyl, haloalkyl such as trifluoromethyl, acyloxy, amino, mono- or di-alkylamino, alkoxyamido, alkoxycarboxylate or an esterified derivative thereof;
R₇ is H or halo;
a is 1-6; and
any of R₁, R₃, R₅, R₈, R₉, R₁₀, R₁₁ and R₁₂ may optionally be substituted one or more times by halo, hydroxy, amino, cyano, nitro, carboxy or oxo;
with the proviso that the compound is not a compound in which R₇ represents H, R₅ represents unsubstituted phenyl, and R₁, R₂, R₃, R₄, R₆ and a are selected from one of the following combinations:

| 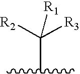 | R₆ | 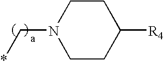 |
|---|---|---|
| 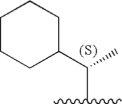 | H | 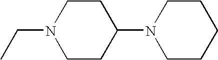 |
| 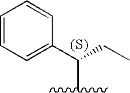 | H | 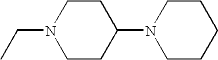 |
| 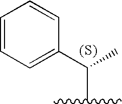 | H | 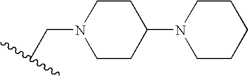 |
| 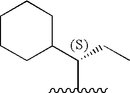 | H | 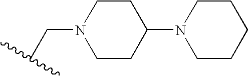 |
| 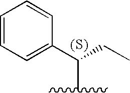 | H | 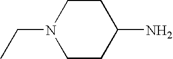 |
| 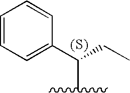 | 7-OMe, 8-Br | 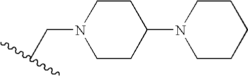 |
| 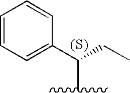 | 7-OMe | 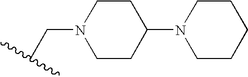 |
| 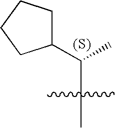 | H | 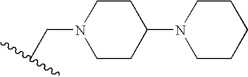 |
| 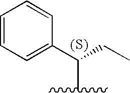 | H | 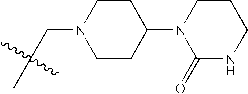 |
| 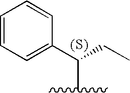 | H | 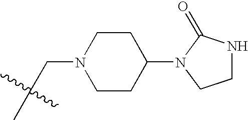 |
| 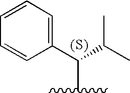 | H | 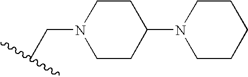 |
| 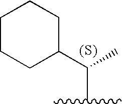 | 7-OMe | 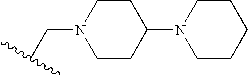 |
| 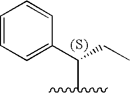 | 7-OH, 8-Cl | 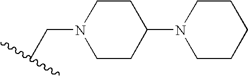 |
| 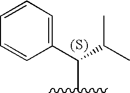 | H | 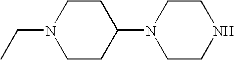 |
| 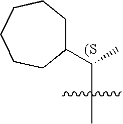 | H | 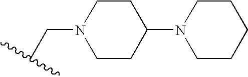 |
| 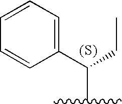 | 7-OH | 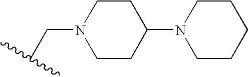 |
| 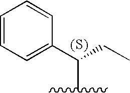 | H | 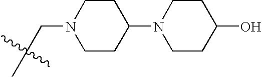 |
| 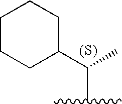 | H | 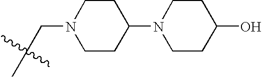 |
| 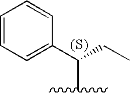 | H | 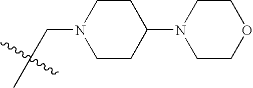 |
| 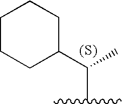 | H | 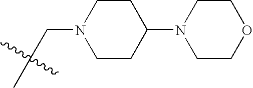 |

2 A compound as claimed in claim 1, wherein R₃ represents methyl, ethyl, iso-propyl, cyclopropyl, hydroxymethyl or hydroxyethyl.

3 A compound as claimed in claim 1 or claim 2, wherein R₈ represents a single bond.

4 A compound as claimed in claim 1 or claim 2, wherein R₈ represents hydroxymethyl.

5 A compound as claimed in any preceding claim, wherein R₉ represents phenyl or cyclohexyl, which phenyl or cyclohexyl is unsubstituted or is substituted by hydroxy or alkoxy such as methoxy or alkoxyalkyl such as methoxymethyl, methoxyethyl, methoxypropyl or methoxybutyl.

6 A compound as claimed in any preceding claim, wherein R₁ is hydrogen.

7 A compound as claimed in any preceding claim, wherein R₅ is unsubstituted phenyl.

8 A compound as claimed in any of claims 1-6, wherein R₅ is phenyl which is substituted one or more times by halo such as fluoro, and/or haloalkyl such as trifluoromethyl.

9 A compound as claimed in any of claims 1-6, wherein R₅ is a heterocyclic ring, such as an unsaturated heterocyclic ring, comprising at least one heteroatom such as S.

10 A compound as claimed in claim 9, wherein R₅ is

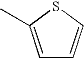

11 A compound as claimed in any preceding claim, wherein R₇ represents hydrogen.

12 A compound as claimed in any preceding claim, wherein R₆ represents hydrogen, or one or more substituents selected from fluoro, chloro, bromo or trifluoromethyl.

13 A compound as claimed in claim 12, wherein each of said one or more substituents is respectively positioned at the 5′, 6′, 7′ or 8′ position around the quinoline ring of said compound.

14 A compound as claimed in any of claims 1-11, wherein R₆ represents one ring substituent, which is hydroxy, alkoxy such as methoxy or ethoxy or a hydroxylated derivative thereof, alkoxycarboxylate such as methoxycarboxylate or ethoxycarboxylate or an esterified derivative thereof such as methoxyethanoate ethoxyethanoate, or alkoxyamido such as methoxyamido or ethoxyamido.

15 A compound as claimed in claim 14, wherein said one ring substituent is located at the 6 or 7 position around the quinoline ring of said compound.

16 A compound as claimed in any preceding claim, wherein R₉ is aryl or heteroaryl, which aryl or heteroaryl is optionally substituted one or more times by hydroxy, alkoxy, or alkoxyalkyl.

17 A compound as claimed in any of claims 1-15, wherein R₉ is cycloalkyl, which cycloalkyl is optionally substituted one or more times by hydroxy.

18 A compound as-claimed in any preceding claim, wherein a is 1, 2 or 3.

19 A compound as claimed in any preceding claim, wherein each of R₁₀ and R₁₁ is hydrogen.

20 A compound as claimed in any of claims 1-18, wherein R₁₀ and R₁₁ together with the nitrogen atom to which they are attached form a saturated heterocyclic ring comprising five or six ring members.

21 A compound as claimed in claim 20, wherein said saturated heterocyclic ring comprises one or more additional nitrogen atoms.

22 A compound as claimed in claim 20 or claim 21, wherein said saturated heterocyclic ring is substituted by oxo.

23 A compound as claimed in claim 16 or claim 17, wherein said saturated heterocyclic ring is substituted by R₁₃ R₁₄ R₁₅, wherein R₁₃ is methyl, ethyl, propyl or butyl, and R₁₅ is H or methyl, ethyl, propyl or butyl.

24 A compound as claimed in claim 23, wherein R₁₄ is C(O)O.

25 A compound as claimed in any preceding claim, wherein R₅ is unsubstituted phenyl, R₆ is H, R₇ is H, and a, R₁, R₂, R₃, and R₄ are selected from the following combinations:

| 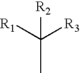 | 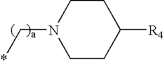 |
|---|---|
| 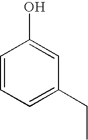 | 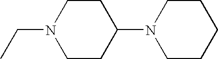 |
| 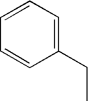 | 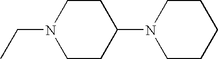 |
| 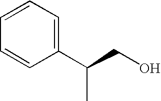 | 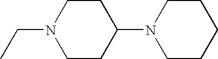 |
| 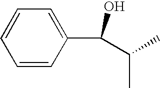 | 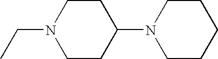 |
| 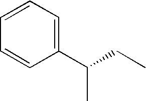 | 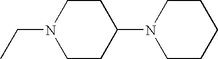 |
| 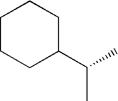 | 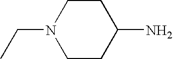 |
| 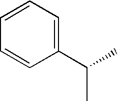 | 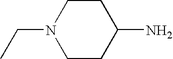 |
| 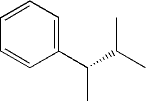 | 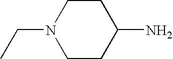 |
| 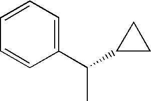 | 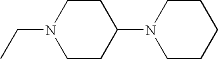 |
| 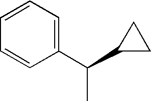 | 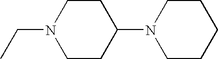 |
| 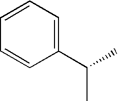 | 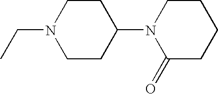 |
| 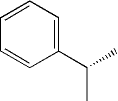 | 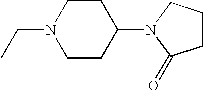 |
| 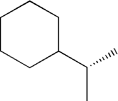 | 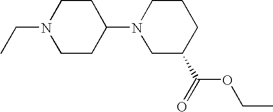 |
| 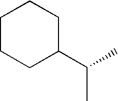 | 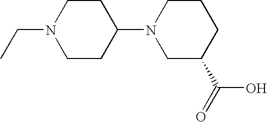 |
| 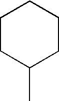 | 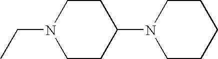 |

26 A compound as claimed in any of claims 1-24, which is selected from the following:

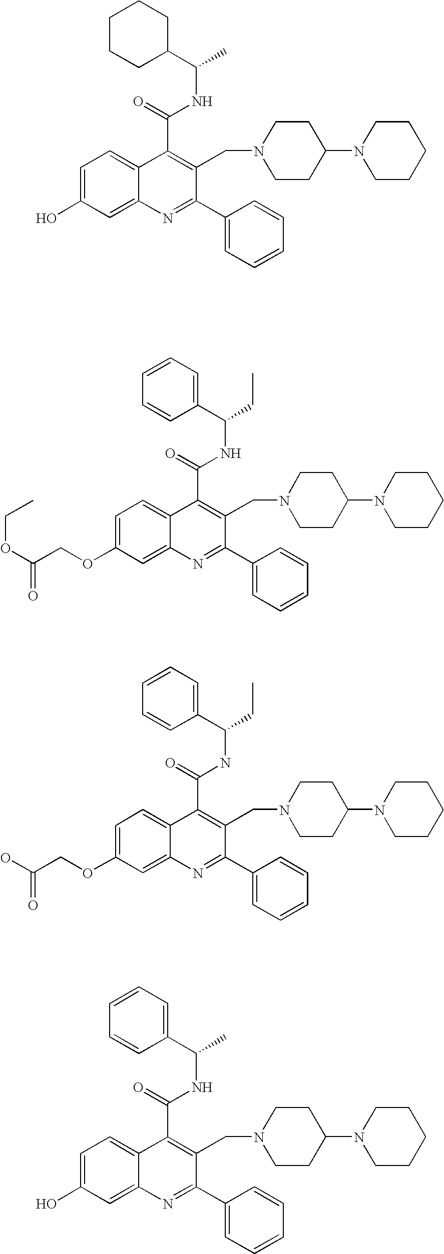

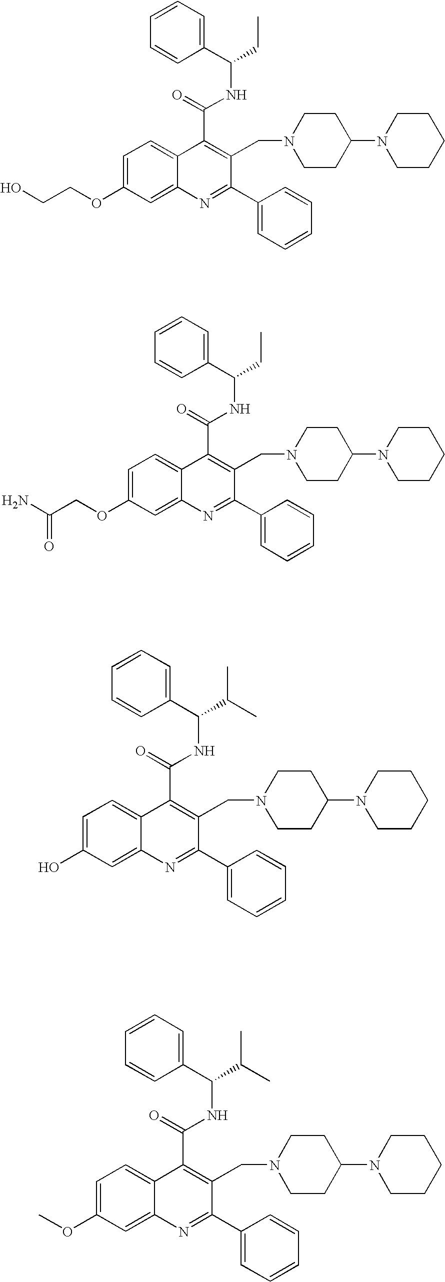

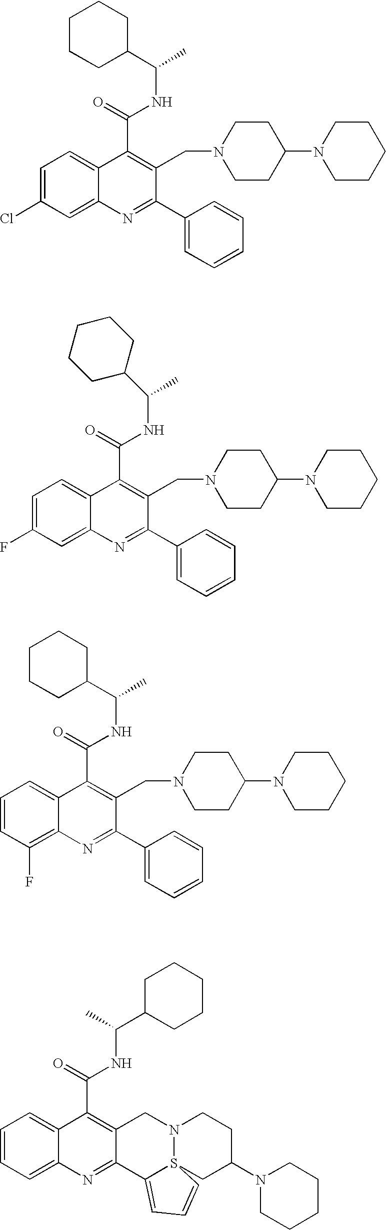

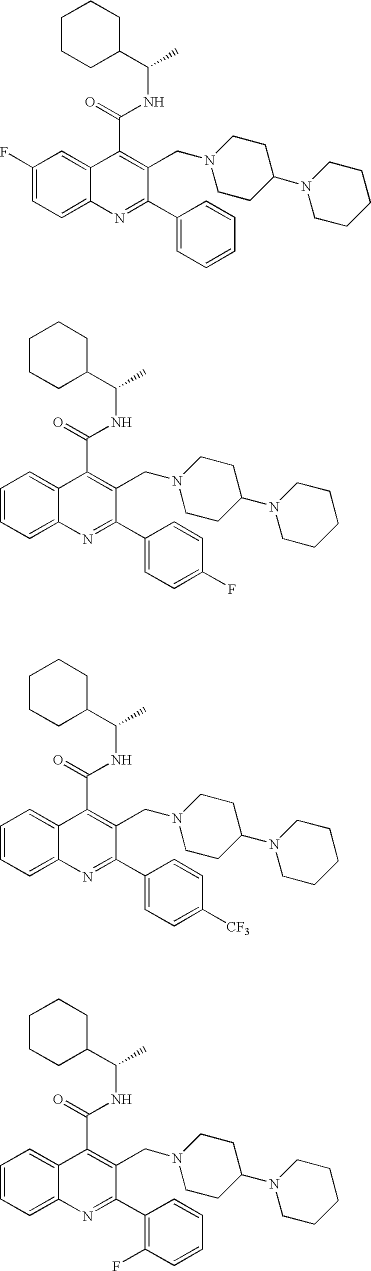

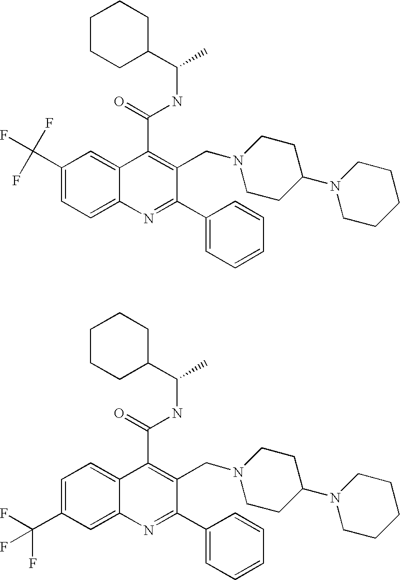

27 A process for the preparation of a compound of formula (I) according to any of claims 1-26, or a salt thereof and/or a solvate thereof, which process comprises reacting a compound of formula (II) or an active derivative thereof: 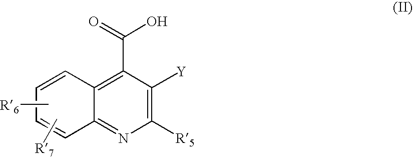
wherein R′₅, R′₆, and R′₇ are R₅, R₆, and R₇ respectively as defined in relation to formula (I) or a group convertible to R₅, R₆, and R₇ respectively, and Y′ is a group of formula (Y) or a group convertible thereto 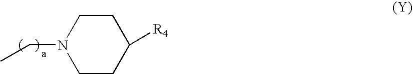
where R₄ is defined as in relation to formula (I), with a compound of formula (III): 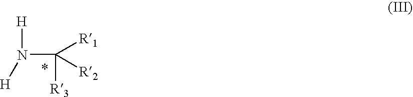
wherein R′₁, R′₂ and R′₃ are R₁, R₂ and R₃ as defined for formula (I) or a group or atom convertible to R₁, R₂ and R₃ respectively; to form a compound of formula (Ib): 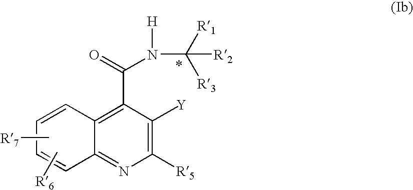
wherein R′₁, R′₂, R′₃, R′₅, R′₆, R′₇ and Y′ are as defined above, and thereafter carrying out one or more of the following optional steps:
(i) converting any one of R′₁, R′₂, R′₃, R′₅, R′₆, R′₇ and Y′ to R₁, R₂, R₃, R₅, R₆, R₇ and Y respectively as required, to obtain a compound of formula (I);
(ii) converting a compound of formula (I) into another compound of formula (I); and
(iii) preparing a salt of the compound of formula (I) and/or a solvate thereof.

28 A pharmaceutical composition comprising a compound of formula (I) according to any of claims 1-26, or a pharmaceutically acceptable salt or solvate thereof, and a pharmaceutically acceptable carrier.

29 A compound of formula (I), or a pharmaceutically acceptable salt or solvate thereof, for use as an active therapeutic substance.

30 A compound of formula (I), or a pharmaceutically acceptable salt or solvate thereof, for the treatment or prophylaxis of the Primary and Secondary Conditions.

31 Use of a compound of formula (I), or a pharmaceutically acceptable salt or solvate thereof, in the manufacture of a medicament for the treatment of the Primary and Secondary Conditions.

32 A method for the treatment and/or prophylaxis of the Primary and Secondary Conditions in mammals, particularly humans, which comprises administering to the mammal in need of such treatment and/or prophylaxis an effective, non-toxic pharmaceutically acceptable amount of a compound of formula (I) or a pharmaceutically acceptable salt or solvate thereof.
